(19) United States
(12) Patent Application Publication
(10) Pub. No.: US 2024/0307179 A1
(43) Pub. Date: Sep. 19, 2024

(54) Retrieval Catheter

(71) Applicant: Cephea Valve Technologies, Inc., Abbott Park, IL (US)

(72) Inventor: Randolf Von Oepen, Aptos, CA (US)

(73) Assignee: Cephea Valve Technologies, Inc., Abbott Park, IL (US)

(21) Appl. No.: 18/591,314

(22) Filed: Feb. 29, 2024

Related U.S. Application Data
(60) Provisional application No. 63/490,319, filed on Mar. 15, 2023.

(51) Int. Cl. A61F 2/24; A61M 25/00; A61M 25/01

(52) U.S. Cl. CPC A61F 2/2427; A61M 25/0012; A61M 25/01; A61M 2025/0175

(57) ABSTRACT

A retrieval catheter for retrieving a partially deployed medical device held at the end of a catheter includes an inner shaft, an intermediate shaft and an outer shaft. The inner shaft engages the catheter in a manner that prevents translation of the inner shaft relative to the catheter. The intermediate shaft is assembled around the inner shaft and includes inner threads that engage with outer threads on the inner shaft such that rotation of the intermediate shaft translates it relative to the inner shaft and the catheter. The outer shaft is assembled around the intermediate shaft and includes a valve cover at its distal end. The valve cover is operatively connected to the intermediate shaft such that translation of the intermediate shaft translates the valve cover. Components of the medical device delivery system are disassembled from the catheter and the retrieval catheter is assembled thereover to retrieve the medical device.

### CROSS-REFERENCE TO RELATED APPLICATIONS

The present application claims the benefit of the filing date of U.S. Provisional Patent Application No. 63/490,319, filed Mar. 15, 2023, the disclosure of which is hereby incorporated herein by reference.

### BACKGROUND OF THE INVENTION

The present disclosure relates generally to devices, systems and methods for delivering an interventional device into a patient for implantation. More particularly, the present disclosure relates to devices, systems and methods for transcatheter delivery of a collapsible prosthetic heart valve to a native valve annulus, to deployment of the prosthetic heart valve, and to retrieval of the prosthetic heart valve should that become necessary.

Interventional medical devices that are collapsible to a relatively small circumferential size can be delivered into a patient less invasively than devices that are not collapsible. For example, a collapsible prosthetic heart valve may be delivered into a patient via a tube-like delivery apparatus such as a catheter, a trocar, a laparoscopic instrument, or the like. This collapsibility can avoid the need for a more invasive procedure such as full open-chest, open-heart surgery, thereby reducing the risks, costs and time associated with an open-heart surgical procedure.

Collapsible prosthetic heart valves typically take the form of a valve structure mounted on a stent. There are two types of stents on which the valve structures are ordinarily mounted: a self-expanding stent and a balloon-expandable stent. Valves with self-expanding stents generally are first collapsed or crimped to reduce their circumferential size, and then loaded into a delivery apparatus. Valves with balloon-expandable stents generally are crimped around a deflated balloon that is mounted to a delivery apparatus.

When a collapsed prosthetic valve has reached the desired implantation site in the patient (e.g., at or near the annulus of the patient's heart valve that is to be replaced by the prosthetic valve), the prosthetic valve can be deployed or released from the delivery apparatus. For balloon-expandable valves, this generally involves assuring the proper location of the valve, and then expanding the balloon positioned within the valve stent. For self-expanding valves, on the other hand, the stent automatically expands as the sheath covering the valve is withdrawn.

There are many considerations in properly deploying a prosthetic heart valve in the native valve annulus. The prosthetic valve should be placed at the same or very nearly the same angle as the native valve. A valve that is off axis could cause turbulent blood flow and/or potential paravalvular leaks. The prosthetic valve also should be implanted so that its center aligns with the center of the native valve. Off-center deployment or implantation of the prosthetic valve could interfere with neighboring valves or the heart's conduction system. Additionally, the prosthetic heart valve should be implanted at the proper depth within the native valve annulus, also to not interfere with the heart's conduction system.

Following the partial deployment of a prosthetic heart valve from the delivery system described herein, it may become necessary to again recapture the prosthetic heart valve. This need may arise when it is determined that a partially deployed prosthetic heart valve needs to be repositioned, or that the prosthetic heart valve has been damaged during partial deployment or otherwise is not functioning properly following partial deployment and needs to be removed from the patient. A retrieval catheter that may be used with the described delivery system and that addresses the need to recapture a partially deployed prosthetic heart valve is described herein.

### BRIEF SUMMARY OF THE INVENTION

The present invention provides a retrieval catheter for retrieving a partially deployed medical device held at the distal end of a catheter. The retrieval catheter includes an inner tubular shaft engageable with the catheter in a manner that prevents translation of the catheter in a longitudinal direction relative to the inner tubular shaft absent rotation of the catheter relative to the inner tubular shaft. The inner tubular shaft has a proximal end, a distal end, and a lumen extending therethrough from the proximal end to the distal end, with an outer surface in a distal portion of the inner tubular shaft having a series of outer threads. The retrieval catheter also includes an intermediate tubular shaft assembled around the inner tubular shaft, the intermediate tubular shaft having a proximal end, a distal end, and an inner surface defining a lumen therethrough from the proximal end to the distal end. The inner surface in a distal portion of the intermediate tubular shaft has a series of inner threads engageable with the outer threads of the inner tubular shaft such that rotation of the intermediate tubular shaft relative to the inner tubular shaft translates the intermediate tubular shaft proximally or distally relative to the inner tubular shaft and the delivery catheter. An outer tubular shaft is assembled around the intermediate tubular shaft and has a proximal end, a distal end, and a lumen therethrough from the proximal end to the distal end. A valve cover at the distal end of the outer tubular shaft is operatively connected to the intermediate tubular shaft such that translation of the intermediate tubular shaft translates the valve cover.

The present invention also provides a method for retrieving a partially deployed medical device held at a distal end of a delivery catheter assembly within a patient, the catheter assembly including an outer sheath having a proximal end and a distal end, a first valve cover at the distal end of the outer sheath, a steering catheter disposed within the outer sheath, an extension catheter disposed within the steering catheter, and a suture catheter disposed within the extension catheter. The medical device is positioned outside of the valve cover and connected to the suture catheter by a plurality of tethers. The method includes removing the extension catheter, the steering catheter, the outer sheath, and the valve cover from the catheter assembly and from the patient; and assembling a retrieval catheter over the suture catheter within the patient. The retrieval catheter includes an inner tubular shaft engageable with the suture catheter in a manner that prevents translation of the suture catheter in a longitudinal direction relative to the inner tubular shaft; an intermediate tubular shaft assembled around the inner tubular shaft, the intermediate tubular shaft being threadedly engaged with the inner tubular shaft; an outer tubular shaft assembled around the intermediate tubular shaft; and a second valve cover at the distal end of the outer tubular shaft, the second valve cover being operatively connected to the intermediate tubular shaft. After the retrieval catheter is assembled over the suture catheter, the method further includes rotating the intermediate tubular shaft relative to the inner tubular shaft to translate the intermediate tubular shaft and the second valve cover distally relative to the suture catheter and the medical device until the second valve cover collapses and covers the medical device; and removing the retrieval catheter, the suture catheter, and the medical device from the patient.

### BRIEF DESCRIPTION OF THE DRAWINGS

A more complete appreciation of the subject matter of the present disclosure and the various advantages thereof can be realized by reference to the following detailed description in which reference is made to the accompanying drawings in which:

FIG. 1A is an illustrative prosthetic heart valve according to the prior art for use in conjunction with the delivery system of the present invention;

FIG. 1B is a perspective view of the frame structure of the prosthetic heart valve of FIG. 1A;

FIG. 2 is a side view of a delivery system configured for delivering, positioning and deploying a prosthetic heart valve, including a handle assembly and a catheter assembly;

FIG. 3A is a transverse cross-sectional view of the catheter assembly of FIG. 2, showing the various components thereof;

FIG. 3B is a longitudinal cross-section of the catheter assembly of FIG. 2, showing the nested arrangement of the various components thereof;

FIG. 4 is a schematic cutaway view of the heart, showing an exemplary approach for delivering the prosthetic heart valve to the mitral valve annulus;

FIG. 5 is a side view of an outer sheath of the catheter assembly, showing the nosecone in a closed position at the distal end thereof;

FIG. 6 is an enlarged view of the distal end of the outer sheath and valve cover shown in FIG. 5;

FIG. 7 is a longitudinal cross-section of the distal end of the outer sheath and the valve cover shown in FIG. 6;

FIG. 8 is an enlarged cutaway view of a highly flexible portion of the outer sheath;

FIG. 9 is a side view of a steering catheter of the catheter assembly, showing various sections thereof;

FIG. 10 is an enlarged partial view of the distal section of the steering catheter of FIG. 9 forming a compound curve shape to enable proper positioning of the catheter assembly relative to the mitral valve annulus;

FIG. 11 is a side view of an extension catheter of the catheter assembly with a distal can structure at its distal end;

FIG. 12A is a side view of a suture catheter of the catheter assembly;

FIG. 12B is an enlarged highly schematic view of a portion of an exemplary suture catheter with a distal suture ring and a plurality of tethers attached thereto;

FIG. 13 is a side view of a nosecone catheter and nosecone of the catheter assembly;

FIG. 14 is a side view of a guidewire for use with the catheter assembly, showing the pigtail distal end;

FIG. 15 is an enlarged perspective view of the delivery system shown in FIG. 2;

FIG. 16 is side view of the delivery system of FIG. 15 mounted to a stabilizer;

FIG. 17 is an enlarged longitudinal cross-section of the distal end of a retrieval catheter for use with components of the catheter assembly of FIG. 2;

FIG. 18 is highly schematic longitudinal cross-section of an alternate embodiment of a shaft of the retrieval catheter of FIG. 17; and

FIG. 19 is an enlarged schematic perspective view showing a mechanism for collapsing the strut frame of a prosthetic valve within a patient.

### DETAILED DESCRIPTION

As used herein, the term “inflow end,” when used in connection with a prosthetic heart valve, refers to the end of the heart valve through which blood enters when the heart valve is functioning as intended, whereas the term “outflow end,” when used in connection with a prosthetic heart valve, refers to the end of the heart valve through which blood exits when the heart valve is functioning as intended. For a prosthetic mitral valve, the inflow end is closest to the left atrium when the heart valve is implanted in a patient, and the outflow end is closest to the left ventricle when the heart valve is implanted in a patient. Further, when used herein in connection with a delivery device, the terms “proximal” and “distal” are to be taken as relative to a user operating the device in an intended manner. “Proximal” is to be understood as relatively close to the user and “distal” is to be understood as relatively farther away from the user. Also as used herein, the terms “substantially,” “generally,” and “about” are intended to mean that slight deviations from absolute are included within the scope of the term so modified.

In the description which follows, a delivery system and the components thereof are described in connection with the delivery, positioning and deployment of a prosthetic mitral valve at the native mitral valve annulus. However, it is to be understood that the delivery system and components described also may be used to deliver, position and deploy prosthetic replacements for other cardiac valves, such as the aortic valve, the pulmonary valve and the tricuspid valve, as well as other medical devices. Exemplary prosthetic heart valves that can be used with the delivery system described herein include the expandable prosthetic heart valves described in U.S. Patent Publication No. 2016/0158000; in U.S. Pat. Nos. 8,870,948 and 10,470,881; and in PCT Publication No. WO 2016/183526, the disclosures of all of which are hereby incorporated by reference herein.

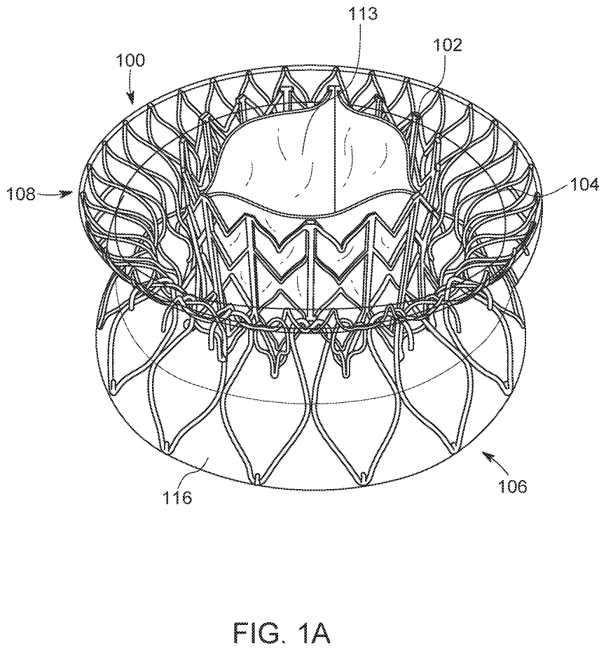

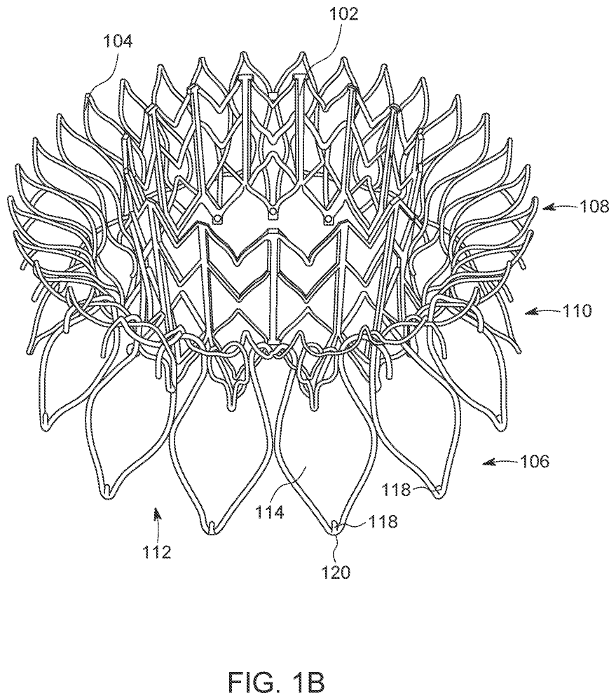

FIG. 1A is a perspective view of an exemplary prosthetic heart valve 100 that may be delivered, positioned and deployed by the delivery system disclosed herein. Prosthetic heart valve 100 may be a prosthetic mitral valve having an expandable and collapsible frame structure that includes an inner strut frame 102 surrounded by an outer anchor assembly 104, as shown in FIG. 1B. Anchor assembly 104, shown in an expanded state, includes an atrial anchor 106 configured to be positioned on the atrial side of the native mitral valve annulus, a ventricular anchor 108 configured to be positioned on the ventricular side of the native mitral valve annulus, and a central portion 110 positioned axially between the atrial anchor and the ventricular anchor. Anchor assembly 104 may have an hourglass shape in the expanded state in that each of atrial anchor 106 and ventricular anchor 108 flares radially outward of central portion 110, such that the central portion defines a waist between the atrial anchor and the ventricular anchor. Strut frame 102 may be positioned radially inward of anchor assembly 104 and may be formed of a plurality of interconnected struts. The radially inner surface of strut frame 102 defines the perimeter of a central opening 112, which enables blood to flow through prosthetic mitral valve 100.

Prosthetic mitral valve 100 includes one or more leaflets 113 that may be secured to strut frame 102 and disposed at least partially in central opening 112. Leaflets 113 are configured to coapt with one another to control blood flow through the prosthetic mitral valve, allowing blood to flow from atrial anchor 106 at the inflow end of the heart valve toward ventricular anchor 108 at the outflow end of the heart valve (the antegrade direction), but substantially blocking blood from flowing in the opposite (retrograde) direction. In some embodiments, one or more skirts or cuffs 116 may partially or fully cover inner and/or outer surfaces of anchor assembly 104 and/or strut frame 102. Such skirts or cuffs may be formed from fabric and/or tissue materials, for example, as may leaflets 113.

Both the atrial anchor 106 and the ventricular anchor 108 of anchor assembly 104 include a plurality of petals 114 that are joined to one another around the circumference of the anchor assembly. When prosthetic mitral valve 100 is in a fully expanded state, the petals 114 on both atrial anchor 106 and ventricular anchor 108 are fully extended radially outward, as shown in FIG. 1B. Prosthetic mitral valve 100 is naturally in an expanded state when no force is applied to petals 114. The petals 114 of anchor assembly 104 may be configured to collapse and/or to reduce the outer diameter of the frame structure when the prosthetic heart valve is loaded into a delivery device. When prosthetic mitral valve 100 is in a collapsed state, the petals 114 on both atrial anchor 106 and ventricular anchor 108 are at least partially collapsed radially inward. Prosthetic mitral valve 100 may be placed in the collapsed state by applying pressure to petals 114 in a radially inward direction.

The petals 114 on atrial anchor 106 or ventricular anchor 108 may include a pin 118 or other attachment member to which tether loops may be connected, as will be described below. Pins 118 may be attached to or formed on some or all of the petals 114 on atrial anchor 106 and/or ventricular anchor 108 and are sized and shaped so that the tether loops remain attached when under tension but are released from the frame structure after the deployment of prosthetic mitral valve 100 within the patient. Pins 118 may be provided, for example, at the apex 120 of each petal 114 on atrial anchor 106. However, this need not be the case and pins 118 may be provided on less than all of the petals 114 of atrial anchor 106, on some or all of the petals of ventricular anchor 108, or at other locations on anchor assembly 104.

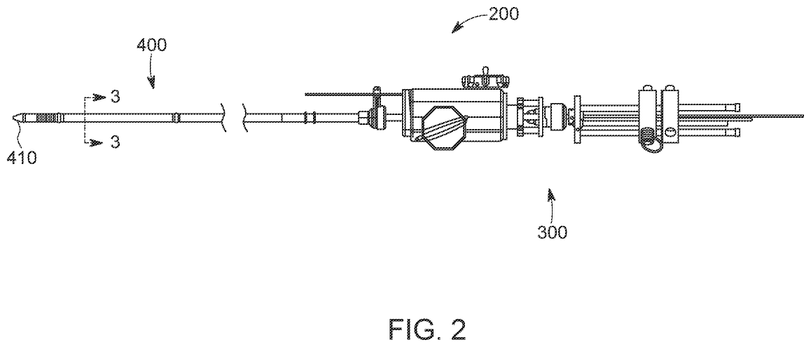

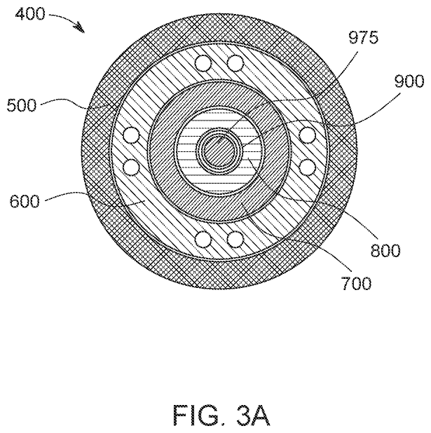

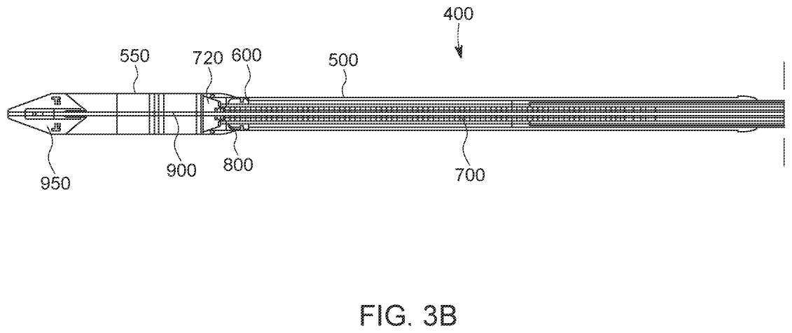

FIG. 2 illustrates an exemplary embodiment of a delivery system 200 for delivering and deploying a prosthetic heart valve at a target location within the patient's heart. Delivery system 200 generally includes a handle assembly 300 and a catheter assembly 400. Catheter assembly 400 extends from a proximal end coupled to handle assembly 300 to an atraumatic tip 410 at a distal end and includes a plurality of catheter and/or hypotube components that are longitudinally slidable relative to one another and that provide different functionality during operation of delivery system 200 to enable effective delivery and deployment of a prosthetic heart valve, such as prosthetic mitral valve 100. As shown in FIG. 3A, which is a cross-sectional view of catheter assembly 400 taken along line 3-3 of FIG. 2, these components include an outer sheath 500, a steering catheter 600, an extension catheter 700, a suture catheter 800, and a nosecone catheter 900, all arranged in a concentric nested relationship. The arrangement of these components, as well as valve cover 550 and nosecone 950, is shown in the longitudinal cross-section of catheter assembly 400 shown in FIG. 3B. As illustrated in the figures, nosecone catheter 900 has a lumen sized to receive a guidewire 975 therein. Each of these components is described in detail below.

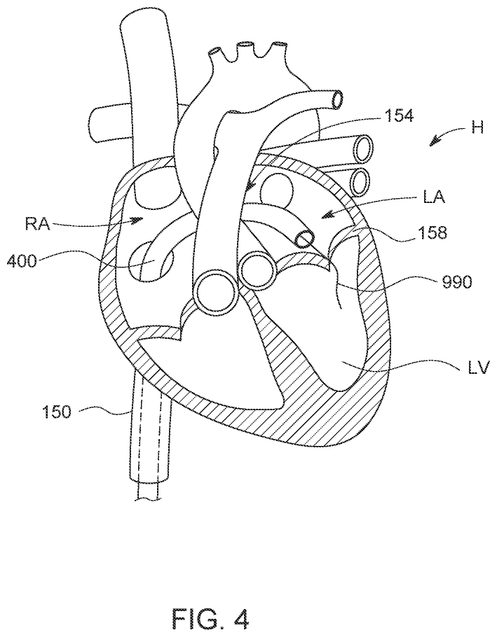

FIG. 4 is a schematic representation of a patient's heart H and a delivery route that may be followed by catheter assembly 400 to reach the native mitral valve annulus 158. Using a transfemoral approach, catheter assembly 400 may be inserted into the patient's femoral vein and advanced through the inferior vena cava 150 to the right atrium RA. In a subsequent transseptal procedure, catheter assembly 400 is advanced through a puncture in intra-atrial septum 154 into the left atrium LA.

In other implementations, such as for procedures associated with a tricuspid valve, catheter assembly 400 may be advanced through the inferior vena cava 150 and into the right atrium RA, where it may then be positioned and used to perform the procedure related to the tricuspid valve. While many of the examples described herein relate to delivery to the native mitral valve annulus, one or more embodiments may be utilized in other cardiac procedures, including those involving the tricuspid valve or other cardiac valves.

Although one preferred method for accessing a targeted cardiac valve annulus is a transfemoral approach, it will be understood that the embodiments described herein may also be utilized where alternative approaches are used. For example, embodiments described herein may be utilized in a transjugular approach, transapical approach, transradial approach or other suitable approaches to the targeted anatomy. For procedures related to the mitral valve or tricuspid valve, the delivery of the prosthetic heart valve or other medical device is preferably carried out from an atrial aspect (i.e., with the distal end of catheter assembly 400 positioned within the atrium superior to the targeted valve). The illustrated embodiments are shown from such an atrial aspect. However, it will be understood that the delivery of the medical devices described herein may also be carried out from a ventricular aspect.

Additional details regarding delivery systems and devices that may be utilized in conjunction with the components and features described herein are described in U.S. Patent Publication Nos. 2018/0028177A1, 2018/0092744A1, and 2020/0155804, the disclosures of which are hereby incorporated by reference herein.

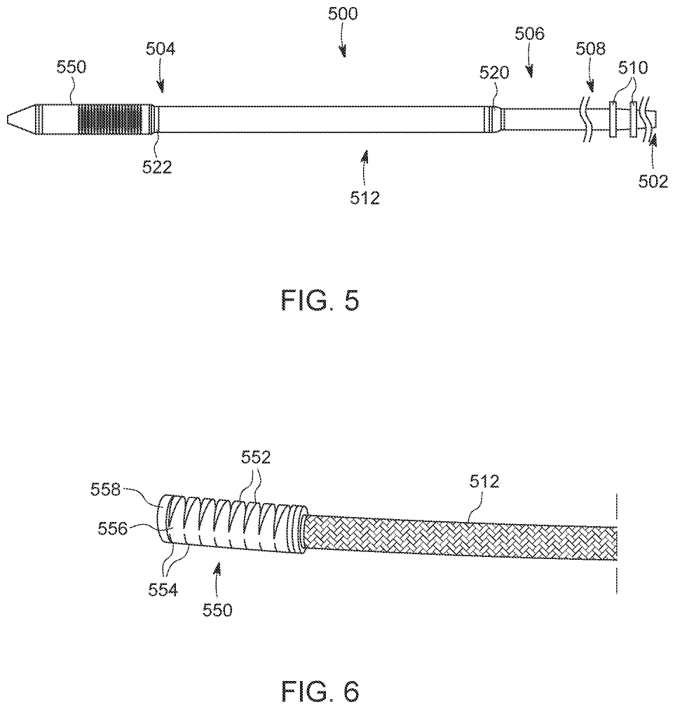

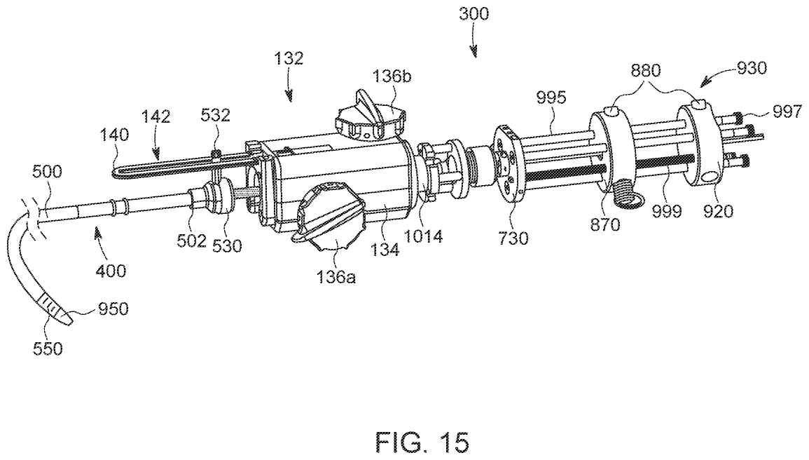

As noted, the outer component of catheter assembly 400 is outer sheath 500, shown in FIGS. 5-7. Outer sheath 500 extends from a proximal end 502 to a distal end 504. A proximal portion 506 of outer sheath 500 may be formed from a stainless-steel hypotube having a plurality of slits (not shown) laser cut in an interrupted spiral pattern along a majority of its length. The slits begin at a spaced distance from the proximal end 502 of outer sheath 500, leaving an uncut portion 508 that enables the proximal end of the outer sheath to be fixedly connected, such as by adhesive, to a hemostasis valve 530 (see, for example, FIG. 15) with a leakproof seal. A pair of fixed rings 510 may be spaced apart from one another around the outside of uncut portion 508. Rings 510 enable outer sheath 500 to be held in place as other components of catheter assembly 400 are advanced or retracted, and also enable the outer sheath itself to be retracted for deploying prosthetic mitral valve 100, as will be discussed further below. Although outer sheath 500 is described as formed from a stainless-steel hypotube, that need not be the case. Outer sheath 500 may alternatively be formed from other metals or metal alloys, including titanium or tantalum, from biologically stable polymers, from biologically stable composites, from a braided polymer shaft (as long as the shaft can transmit the axial forces needed to unsheathe a valve), and the like.

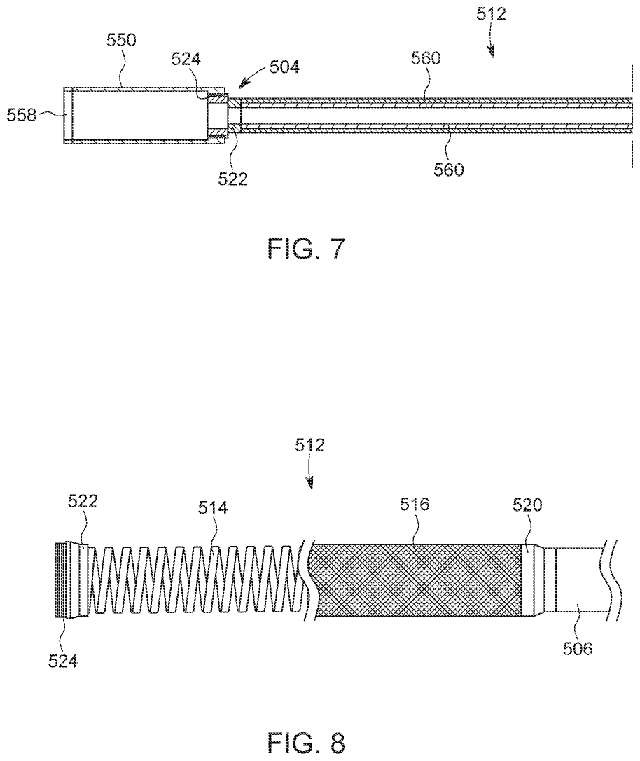

FIGS. 6 and 7 illustrate a distal portion of outer sheath 500 and a connected valve cover 550. The distal portion of outer sheath 500 includes a highly flexible portion 512 extending from proximal portion 506 to the distal end 504 of the outer sheath and having a sufficient length to surround and extend along that portion of catheter assembly 400 that is designed to bend and reorient to navigate through a patient's vasculature and heart to reach mitral valve annulus 158 for deployment of the prosthetic heart valve. In some embodiments, flexible portion 512 may have an inner coiled layer 514 formed from a coiled wire and an outer braided sleeve 516 covering the coiled layer (sometimes collectively referred to as “coil/braid portion 514/516”), as shown in FIG. 8. Coiled layer 514 is formed so that there are spaces between adjacent turns of the coil. In some embodiments, the spaces between adjacent turns of the coil are greater than the cross-section of the wire forming the coil. In such embodiments, longitudinally compressing the coil to eliminate the spaces between adjacent turns can reduce the overall length of coiled layer 514 by about 50% to about 70% or more of its original length. Braided sleeve 516 may be fixedly connected at its proximal and distal ends to coiled layer 514 by soldering through the filaments of the braid. The resultant coil/braid portion 514/516 exhibits a high degree of flexibility.

A stainless-steel ring 520 may be laser welded to the proximal end of coiled layer 514. As ring 520 is not covered by braided sleeve 516, it enables the proximal end of flexible portion 512 to be laser welded to the proximal portion 506 of outer sheath 500. Another stainless-steel ring 522 may be laser welded to the distal end of coiled layer 514, followed by another stainless-steel ring 524 that has a slightly larger diameter and that is externally threaded. The external threads provide for a threaded connection of valve cover 550 to coil/braid portion 514/516, and the slightly larger diameter of ring 524 enables the connection to be made without any exposed edges. A threaded connection enables valve cover 550 to be removably connected to outer sheath 500. Additionally, a threaded connection facilitates the joining of dissimilar metals, such as titanium valve cover 550 to a stainless-steel outer sheath 500. However, when possible, the valve cover alternatively may be laser welded to the outer sheath. In a further variant, braided sleeve 516 may be connected to coiled layer 514 by adding the small rings 520 and 522 over the proximal and distal ends, respectively, of the coil/braid portion 514/516 and laser welding all three components together simultaneously.

As outer sheath 500 is retracted to deploy the prosthetic heart valve, internal friction between collapsed prosthetic mitral valve 100 and the valve cover 550 in which it is housed will inhibit the retraction of coiled layer 514, causing the individual coils to slightly separate farther from one another and braided sleeve 516 to lengthen. As braided sleeve 516 lengthens, its diameter will collapse around coiled layer 514, much like a Chinese finger trap, preventing further lengthening of coil layer 514 while still retaining the flexibility of flexible portion 512. Further, the collapse of braided sleeve 516 around coiled layer 514 keeps flexible portion 512 round when it is bent, preventing it from assuming an oval shape that could make it difficult to retract outer sheath 500 to deploy the prosthetic heart valve.

Valve cover 550 defines a compartment for housing prosthetic mitral valve 100 in a compressed, pre-deployed state during intravascular delivery of the prosthetic valve to the targeted cardiac site. Valve cover 550 may be in the form of a generally cylindrical very thin-walled tube of a hard, lightweight metal, such as titanium, having an inner diameter and length sized to receive the prosthetic mitral valve in a collapsed condition. In some embodiments, valve cover 550 may have a wall thickness of between about 0.10 mm (or thinner) and about 0.50 mm, or between about 0.30 mm and about 0.40 mm. A series of internal threads (not shown) may be cut at the proximal end of valve cover 550 for engagement with the external threads of ring 524 at the distal end of outer sheath 500.

Titanium is sufficiently inert that it will not interact with or contaminate, or is less likely to interact with or contaminate, the nitinol forming the frame 102 of prosthetic mitral valve 100 as the valve is retracted into and held within valve cover 550. A series of V-shaped cuts 552 may be laser cut along one side of valve cover 550, and a series of slits 554 may be laser cut along the diametrically opposed side of the valve cover, leaving a pair of continuous longitudinal spines 556 along opposite sides of the valve cover. The series of cuts 552 and slits 554 enable valve cover 550 to bend in a single plane. As valve cover 550 bends, cuts 552 will collapse, while slits 554 will open. Preferably, cuts 552 and slits 554 are sufficient to enable valve cover 550 to bend about 75° or more; preferably, about 90° or more, or about 150° or more, so that the valve cover can be pulled over the distal bend of the steering catheter as it is retracted during deployment of prosthetic mitral valve 100. Cuts 552 and slits 554 are not formed along a distal section of valve cover 550 so as to not interfere with the protruding tines of the prosthetic mitral valve 100 as the prosthetic valve is retracted into the valve cover. Although the foregoing describes the use of titanium to form valve cover 550, other sufficiently strong and inert metals and metal alloys may also be used to form the valve cover.

The spines 556 formed along opposite sides of valve cover 550 between cuts 552 and slits 554 provide the valve cover with sufficient tensile strength to withstand the retraction of outer sheath 500 during deployment of a prosthetic heart valve. The distal end of valve cover 550 may have a number of external threads (not shown) to attach a loading funnel to the valve cover. After prosthetic mitral valve 100 has been loaded into valve cover 550 and the funnel has been removed, a tantalum ring 558 may be threaded onto the distal end of the valve cover to cover the threads and ensure a smooth surface. When formed from a very radiopaque material such as tantalum, platinum, iridium, gold or other materials with a high atomic number, ring 558 helps the user locate the end of valve cover 550 under x-ray imaging to assure the proper location and orientation of the valve cover for deployment of the prosthetic valve. Rather than incorporating threads at the proximal end of valve cover 550 for a removable connection to outer sheath 500 and at the distal end of the valve cover for the removable connection of ring 558, other removable connecting mechanisms may be used, including snap connections, bayonet connections and the like.

The entire length of outer sheath 500 and valve cover 550 may be covered by a flexible, elastic, fluid-impermeable jacket 560 to seal the slits in the proximal portion 506 and the coil/braid portion 514/516 in the flexible portion 512 of the outer sheath and the cuts 552 and slits 554 in the valve cover. Alternatively, jacket 560 may extend only from valve cover 550 to a location just proximal of coil/braid portion 514/516, and another fluid-impermeable jacket may be applied over the slits in the proximal portion 506 of outer sheath 500. In yet another arrangement, jacket 560 may be interposed between the coiled layer 514 and braided sleeve 516 of outer sheath 500, with other fluid-impermeable layers over valve cover 550 and the slits in the proximal portion 506 of the outer sheath. Any of these arrangements may be employed, as long as a liquid-tight structure results. The purpose of the fluid-impermeable layer or layers is to enable the entirety of catheter assembly 400 to be flushed with a saline solution to remove all air. Jacket 560 may be formed from a tube of Texin® synthetic resin available from Covestro LLC, or from another elastic material. After the application of jacket 560, the proximal and distal ends of the jacket may be bonded to the underlying structure using thermal adhesives, UV-bonded adhesives, other types of adhesives, thermal boding, heat shrinking or other techniques.

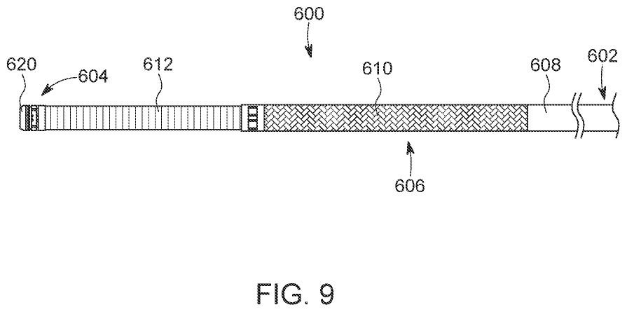

Steering catheter 600 may be nested within outer sheath 500 and may extend from a proximal end 602 connected to steering catheter handle 132 to a distal end 604. Steering catheter 600 is configured to be selectively curved to facilitate navigation through the patient's vasculature and portions of the heart. Referring to FIG. 9, steering catheter 600 may be formed from a stainless-steel hypotube 606 having a proximal section 608, an intermediate section 610 and a distal section 612. The proximal section 608 of hypotube 606 may be uncut to provide steering catheter 600 with a desired amount of stiffness, torqueability and pushability, as well as the ability to form a leakproof connection with steering catheter handle 132. Intermediate section 610 and distal section 612, on the other hand, may be laser cut in a way that enables each section to have sufficient flexibility to achieve a desired bending radius. Intermediate section 610 may be laser cut in an interrupted spiral pattern along its length, much like the proximal portion 506 of outer sheath 500, and may include an outer metal braided layer. Distal section 612 may include a plurality of V-shaped or island cuts and slits, similar to those in valve cover 550, formed along opposite sides of hypotube 606, with each successive pair of cuts and slits being offset in the circumferential direction from an adjacent pair of cuts and slits by 90°. This arrangement of cuts and slits enables distal section 612 to be deflected within one, two or three planes orthogonal to one another. Although steering catheter 600 has been described above with interrupted spiral cuts in intermediate section 610 and V-shaped cuts and slits in distal section 612, other cutting patterns and shapes are possible. For example, a series of narrow slits may be formed along one side of steering catheter 600, the slits enabling longitudinal forces to be transmitted along the catheter and allowing expansion of the catheter when it is deflected in a direction opposite the slits. A series of wider island cuts may be formed along the side of steering catheter 600 opposite the slits, the island cuts allowing compression of the catheter when it is deflected in the direction of the island cuts. The island cuts may be formed so that they progressively change in size in the length direction of steering catheter 600. For example, the island cuts may progressively get smaller in the distal direction along the length of steering catheter 600, providing a gradient bend that gradually increases in deflection at successively more proximal sections.

A steering or tip ring 620 may be secured to the distal end 604 of steering catheter 600. To selectively control the curvature of the distal section 612 of steering catheter 600, the steering catheter may be provided with a plurality of tension cables (not shown). The tension cables may travel from steering catheter handle 132 through a plurality of polymer tubes to the tip ring 620 at the distal end 604 of steering catheter 600. In one embodiment, steering catheter 600 may include four polytetrafluoroethylene (PTFE) tubes equally spaced at 90° intervals around the circumference of hypotube 606. In another embodiment, steering catheter 600 may include four pairs of PTFE tubes (a total of eight tubes), with the pairs of tubes equally spaced at 90° intervals around the circumference of hypotube 606. Any number of these tubes may be provided depending on the various directions of deflection that may be desired. Tip ring 620 may include a plurality of apertures spaced in pairs around its circumference, with a seat and a cutout longitudinally aligned distally of each aperture pair. Each tension cable may travel distally through one of the tubes, through one of a pair of apertures in tip ring 620, through a cutout and around a seat, and then proximally though the other aperture in the pair. At that point, the tension cable may travel proximally through the same tube (when there are four tubes) or through another tube (when there are eight tubes) back to the proximal end 602 of steering catheter 600 where the cable ends are attached to and controlled by steering catheter handle 132. One or more tension cables may additionally or alternatively be coupled at its distal end to the intermediate section 610 of steering catheter 600 to provide the ability to selectively control the curvature of the intermediate section. A polymer layer formed, for example, from Pebax, may be provided as an outer layer surrounding a portion of tip ring 620 and steering catheter 600, including the tubes guiding the tension cables, to produce a liquid-tight structure and impart substantial lubricity to the steering catheter.

One of the functions of delivery system 200 is to position the distal tip of catheter assembly 400 so that prosthetic mitral valve 100 can be deployed in the proper location. This may be accomplished by bending steering catheter 600 in two separate planes via a transseptal approach.

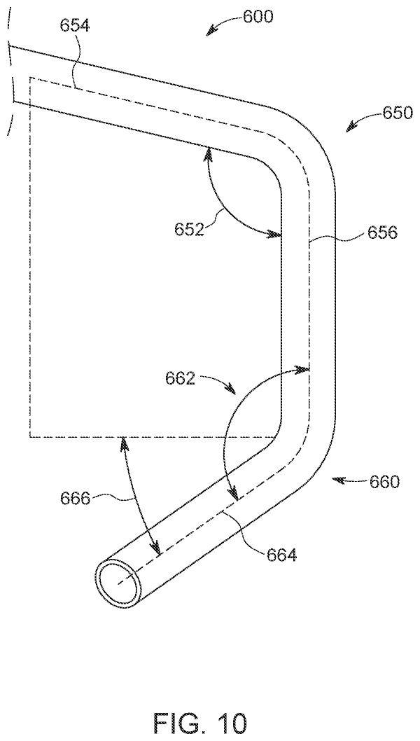

FIG. 10 illustrates an example of a series of complex bends that the distal section 612 of steering catheter 600 may make during the delivery, recapture or repositioning of a prosthetic mitral valve. While accessing the mitral valve annulus, the distal section 612 of steering catheter 600 may be steered in at least two planes of motion that may be substantially perpendicular to one another. In the illustrated example, steering catheter 600 has a first bend 650 with a first bend angle 652 measured between the longitudinal axis 654 of a first portion of the steering catheter and the longitudinal axis 656 of a second portion of the steering catheter. In some embodiments, the first bend angle 652 may be between about 40° and about 150°, more often between about 90° and about 120°, or about 105°. Steering catheter 600 may also have a second bend 660 with a second bend angle 662 between longitudinal axis 656 and the longitudinal axis 664 of a third portion of the steering catheter. In one embodiment, the second bend angle 662 may be between about 45° and about 135°, or about 60°. The second bend 660 may also have a rotational angle 666 relative to the plane in which the first longitudinal axis 654 and the second longitudinal axis 656 lie. In other words, the rotational angle 666 reflects the amount of rotation of the third longitudinal axis 664 relative to the direction of the first bend 650. In some embodiments, the rotational angle 666 may be between about 45° and about 135°, or about 60°. In some embodiments, steering catheter 600 may achieve bend angles of up to 180°.

The distal-most section of steering catheter 600 preferably has a relatively straight section. This may be manifest as an uncut section at the distal end 604 of steering catheter 600. The uncut, relatively straight section allows the components advancing past the distal end 604 of steering catheter 600 to continue along a straight path. For example, by pointing the distal end 604 of steering catheter 600 directly at the mitral valve annulus, the components advancing distally beyond the steering catheter (such as, for example, extension catheter 700) will continue on a straight trajectory toward/through the annulus. Moving proximally from the straight, uncut section, the bend in steering catheter 600 may form gradually before increasing to form the full bend. Other methods of achieving a straight section at the end of steering catheter 600 are also possible. In one example, can 720 described below can be attached directly to the tip ring 620 of the steering catheter, and the can may be provided with a straight section. In another example, the tip ring 620 of the steering catheter itself may be provided with a straight section.

To provide effective steering and positioning at the mitral annulus, the distal section 612 of steering catheter 600 may be cut with a pattern that enables a bending radius of about 15 mm or less, or between about 5 mm and about 15 mm. The intermediate section 610 of steering catheter 600 may be cut to enable a bending radius of between about 30 cm and about 45 cm. The proximal section 608 of steering catheter 600 may remain uncut to ensure that the steering catheter has sufficient stiffness, torquability and pushability to effectively operate, and to enable a leakproof connection between the steering catheter and steering catheter handle 132.

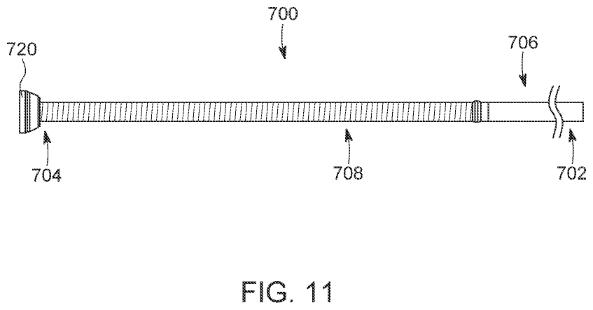

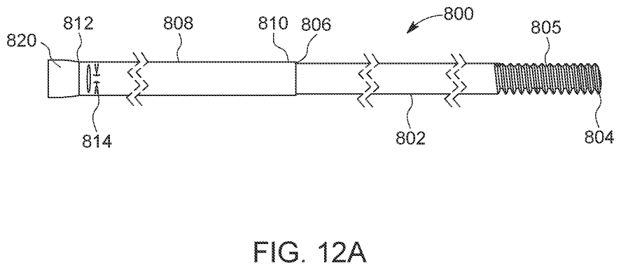

Nested within steering catheter 600 is an extension catheter 700, one embodiment of which is shown in FIG. 11. Alternate embodiments of extension catheter 700 having a structure and features other than those described herein may also be used. Extension catheter 700 may have to withstand relatively high compression forces during the deployment of a prosthetic heart valve. For example, during the release of prosthetic mitral valve 100 by the retraction of outer sheath 500, the countervailing compression force on extension catheter 700 may be on the order of about 30 to 50 lbs. Extension catheter 700 must also have sufficient flexibility to allow for proper deflection and curvature to achieve a desired position in the mitral valve annulus. A coil structure, such as in a distal section of extension catheter 700, is beneficial because it has high flexibility and is also able to withstand high compression forces. In some embodiments, the coil may be a flat wire stacked coil (i.e., a coil in which adjacent turns contact one another with no gaps between them), which has been found to provide an effective balance between flexibility and compressive strength.

Referring to FIG. 11, extension catheter 700 extends from a proximal end 702 to a distal end 704, with a proximal section 706 adjacent the proximal end and a distal section 708 adjacent the distal end. The proximal section 706 of extension catheter 700 may be formed from a stainless-steel hypotube having a laser cut interrupted spiral pattern of slits (not shown) beginning at a spaced distance from proximal end 702. The distal section 708 of extension catheter 700 may be formed with a tri-coil structure. That is, distal section 708 may be formed with three layers of wire coils, with the inner and outer layers being wound in the same direction, and the middle layer being wound in a direction opposite that of the inner and outer layers. In one example, the inner coil may consist of a single wire or filar wound into a coil; and the middle and outer layers may each consist of a bundle of six filars arranged side-by-side and wound into a coil. Each of the individual filars may have a rectangular cross-section. Layers with coils having different numbers of filars are also contemplated. The three coils are coextensive and are welded to one another at their ends, as well as welded to proximal section 706. The tri-coil structure provides distal section 708 with enough flexibility to enable extension catheter 700 to be advanced distally through catheter assembly 400 when the distal end of steering catheter 600 has been deflected into a tight curve to align prosthetic mitral valve 100 with mitral annulus 158. As a result, extension catheter 700 is better able to maintain the position of prosthetic mitral 100 valve relative to native mitral annulus 158 as catheter assembly 400 is manipulated to deploy the prosthetic valve.

Extension catheter 700 includes a can structure 720 that may be laser welded to distal end 704. Can 720 is configured to constrain and hold at least the atrial petals 114 of prosthetic mitral valve 100. Without such constraint, atrial petals 114 may bend outwardly under compression when valve cover 550 is retracted, locking the prosthetic mitral valve inside the valve cover and making it more difficult to unsheathe or re-sheathe it. Can 720 also provides an effective structural surface to act as a counterforce to maintain the prosthetic heart valve in a proper pre-deployed position when outer sheath 500 is retracted.

A suture catheter 800 may be positioned within extension catheter 700. Generally, suture catheter 800 must have a sufficiently flexible distal end that is able to accommodate the tight curves at the distal end of steering catheter 600. In addition, suture catheter 800 must withstand substantial tension during the loading of prosthetic mitral valve 100 into valve cover 550 and must maintain axial tension on the prosthetic mitral valve prior to deployment. By maintaining such axial tension, suture catheter 800 may aid in maintaining the atrial petals 114 of the prosthetic valve within can 720 and the position of the prosthetic valve within catheter assembly 400, as will be described further below.

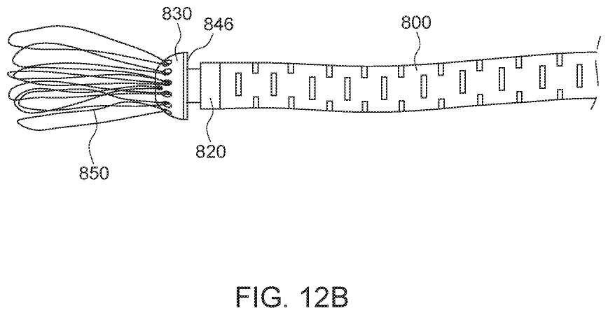

One embodiment of a suture catheter 800 is shown in FIG. 12A, although embodiments of the suture catheter having alternative features and constructions are contemplated herein. Suture catheter 800 has a relatively long proximal portion 802 that may be formed from a stainless-steel hypotube having a proximal end 804 and a distal end 806. A portion of the hypotube adjacent proximal end 804 may include external threads 805 for attachment to the internal threads of a suture catheter control 870, as described below, although other mechanisms for attaching the suture catheter to the suture catheter control may also be used, including a snap fit connector, retaining clip, bayonet connector, and the like. The proximal portion 802 of suture catheter 800 may include an interrupted spiral pattern of slits cut along its length, the slits terminating at a spaced distance from external threads 805. Suture catheter 800 may also have a relatively short distal portion 808 formed from a length of hypotube having a proximal end 810, a distal end 812 and a pattern of dog bone slits 814 laser cut along its length. The dog bone slits formed in the distal portion 808 of suture catheter 800 enable the distal portion to form tight bends while still withstanding a high degree of tension. A thin-walled stainless-steel ring (not shown) may be laser welded at one end to the proximal end 810 of the distal portion 808 of suture catheter 800 so that it protrudes therefrom. The distal end 806 of the proximal portion 802 of the suture catheter may then be assembled over the protruding portion of the ring, which aligns proximal portion 802 with distal portion 808 so they can be joined together by a seam weld.

A proximal suture ring 820 may be laser welded to the distal end 812 of the distal portion 808 of suture catheter 800. Proximal suture ring 820 may be internally threaded at its distal end to mate with the external threads of a distal suture ring 830, shown in FIG. 12B. A preferred distal suture ring 830 for use with catheter assembly 400 is described in U.S. Provisional Application No. 63/228,269, the disclosure of which is hereby incorporated by reference herein. However, the present disclosure contemplates the use of alternative embodiments of the distal suture ring that may have a different construction and different features. A plurality of tethers 850 connected to distal suture ring 830 are intended to releasably hook onto the pins 118 of prosthetic mitral valve 100. As a result, as suture catheter 800 is retracted relative to outer sheath 500, tension will be applied through distal suture ring 830 and tethers 850 to the prosthetic mitral valve, drawing it into the can 720 of extension catheter 700 and valve cover 550. A free-floating polytetrafluoroethylene tube (not shown) may line the lumen of distal portion 808, covering any sharp edges and weld lines resulting from the assembly of the components and providing a low-friction surface to facilitate the sliding of nosecone catheter 900 within suture catheter 800. In addition, a low friction tube (not shown) may be heat shrunk around the outside of distal portion 808 and the spiral cut section of proximal portion 802 to reduce the friction between the outside diameter of suture catheter 800 and the inside diameter of extension catheter 700. This low friction tube may be formed from polytetrafluoroethylene or, more preferably, from a fluorinated ethylene propylene, although other low friction materials may be used.

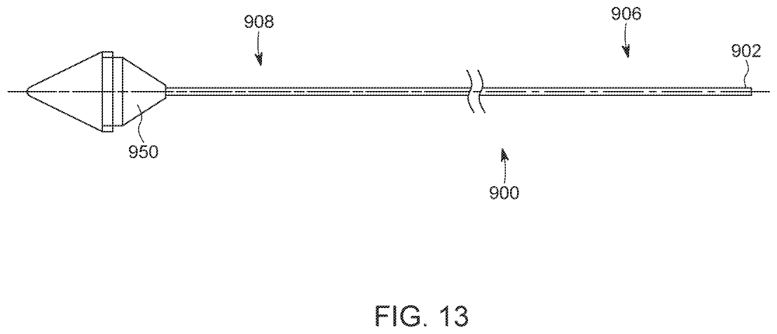

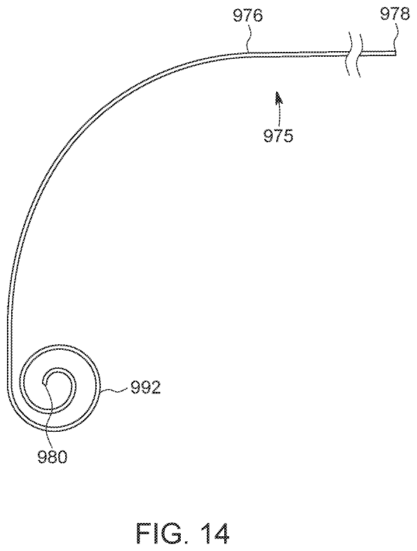

Nosecone catheter 900, an embodiment of which is illustrated in FIG. 13, may be positioned within the lumen of suture catheter 800 and may comprise the innermost component of catheter assembly 400. However, the use of alternative embodiments of the nosecone catheter that may have a different construction and different features is contemplated herein. Nosecone catheter 900 has a lumen therethrough configured to receive guidewire 975. For example, the lumen of nosecone catheter 900 may have a diameter of about 0.037 inches so as to be compatible with a standard 0.035-inch guidewire, although other sizes may be utilized according to particular application needs. Nosecone catheter 900 extends from a proximal end 902 to a distal end (not shown) that resides within a nosecone 950. A proximal portion 906 of nosecone catheter 900 may be formed from a very thin-walled stainless-steel hypotube that, because of its thin wall and small diameter, is flexible. However, nosecone catheter 900 may also be formed from a polymer, such as polyimide, nylon, etc. Adjacent its distal end, nosecone catheter 900 may have a distal portion 908 that includes 2-3 concentric layers of coiled wire laser welded to the hypotube of proximal portion 906. The coiled structure of distal portion 908 provides much greater flexibility than that of proximal portion 906, enabling nosecone catheter 900 to follow the tight bends of steering catheter 600 within the restricted confines of the heart. The innermost coil of distal portion 908 may be wound in a first direction and the overlying coil may be wound in the opposite direction. In addition to providing improved flexibility, the coiled structure of distal portion 908 provides good resistance to any tension forces applied to nosecone catheter 900, thereby minimizing the possibility of the nosecone catheter breaking under such forces. To further improve the strength of distal portion 908 under tension, a third coil layer may be applied over the first two coiled layers, with the third coil layer being wound in the direction opposite that of the underlying coil layer (i.e., in the same direction as the innermost coil). Nosecone 950 may be removably attached to the distal end of nosecone catheter 900, such as by complementary threads, snap fit, bayonet connection or other known releasable connectors. By making nosecone 950 detachable from nosecone catheter 900, prosthetic mitral valve 100 can be loaded into a fully assembled catheter assembly 400, including nosecone catheter 900. That is, the distal end of nosecone catheter 900 can be inserted through prosthetic mitral valve 100 during the loading procedure, without having to slide the prosthetic mitral valve over the entire length of the nosecone catheter from its proximal end 902. Nosecone 950 may have a lumen through its length that is similar in diameter to that of the lumen through nosecone catheter 900. The lumens in nosecone 950 and nosecone catheter 900 coaxially align with one another to slidably receive guidewire 975. At least some embodiments of nosecone 950 that may be used with delivery system 200 are described in detail in U.S. Patent Publication No. 2020/0323634, the disclosure of which is hereby incorporated by reference herein.

Guidewire 975 is a thin wire that is used to guide catheter assembly 400 from the insertion site in the patient to the mitral valve annulus 158 at which the prosthetic mitral valve is to be deployed. To enable catheter assembly 400 to track all the way to mitral valve annulus 158, it may be preferable for the distal tip of guidewire 975 to be advanced into left ventricle LV. Guidewire 975 preferably is formed of a superelastic material, such as nitinol, providing the guidewire with a high degree of flexibility and kink-resistance in order to accommodate the tight turns encountered while advancing from the femoral vein and inferior vena cava 150, through septum 154 and mitral valve annulus 158, to left ventricle LV. One embodiment of a guidewire 975 for use with catheter assembly 400 is shown in FIG. 14 and described in U.S. Patent Publication No. 2020/0155803, the disclosure of which is hereby incorporated by reference herein, although guidewires having different constructions may also be used. Guidewire 975 extends from a proximal end 978 to a distal end 980, and may include a distal portion 992 with a pigtail shape, as shown in FIG. 14. The curved configuration of the pigtail shape may prevent guidewire 975 from damaging cardiac or other tissue as it is advanced to left ventricle LV. Guidewire 975 may have a length between about six ft. (183 cm) and about 15 ft. (457 cm) and a maximum diameter between about 0.014 in. and about 0.035 in.

As mentioned previously, catheter assembly 400 is connected at its proximal end to handle assembly 300, shown in FIG. 15, which consists of steering catheter handle 132 and a series of end rings, caps and other structures, each of which may be connected at or near the proximal end of one of the components of catheter assembly 400. The proximal end 602 of steering catheter 600 may be fixedly connected to the housing 134 of steering catheter handle 132. Steering catheter handle 132 may include one or more controls 136a, 136b to which the proximal free ends of the steering catheter tension cables are connected. Manipulation of controls 136a, 136b adjusts the tension in the tension cables to deflect or straighten the distal section 612 (and, in some embodiments, the intermediate section 610) of steering catheter 600. Manipulation of one control 136a may deflect the distal section 612 of steering catheter 600 in a first plane, and manipulation of the other control 136b may deflect the distal section of the steering catheter in a plane orthogonal to the first plane, or in a plane otherwise transverse to the first plane, depending on the pattern of laser cuts in the steering catheter. Hence, controls 136a, 136b can be adjusted in tandem to position the distal end of catheter assembly 400 and valve cover 550 at a desired position and orientation relative to mitral valve annulus 158. Although controls 136a, 136b are shown in FIG. 15 as knobs, alternative embodiments may additionally or alternatively include one or more buttons, sliders, ratcheting mechanisms, or other suitable controls capable of adjusting the tension in the steering catheter tension cables to provide steering. Illustrative structures that can be used as part of steering catheter handle 132 and/or steering catheter 600 are described in U.S. Pat. No. 7,736,388, the disclosure of which is hereby incorporated by reference herein. However, it is contemplated that mechanisms that operate in a different way and that have a different structure than steering catheter handle 132 may also be used. Since steering catheter 600 is positioned within outer sheath 500, deflection of the steering catheter causes a corresponding deflection/steering of the outer sheath.

In the embodiment shown in FIG. 15, outer sheath 500 is assembled over steering catheter 600 and extends from its distal end 504 to its proximal end 502 which, as noted above, is fixedly connected to an outer sheath hemostasis valve 530 spaced distally of steering catheter handle 132. This positioning enables outer sheath 500 to be advanced distally relative to steering catheter 600 and retracted proximally relative to the steering catheter until hemostasis valve 530 abuts steering catheter handle 132. Although the foregoing describes steering catheter handle 132 as limiting the proximal retraction of outer sheath 500, that need not be the case, and the proximal retraction of the outer sheath can be limited by other features, for example, other components of catheter assembly 400 or features of stabilizer 1000 described below. The luer lock 532 of hemostasis valve 530 extends into and is movable along an elongated slot 142 in a rigid keyway 140 fixedly connected to, and projecting distally from, steering catheter handle 132. The engagement of luer lock 532 in slot 142 also rotationally keys steering catheter 600 to outer sheath 500. As noted above, outer sheath 500 may be laser cut to produce preferred bending directions. However, the bending of outer sheath 500 relies on the bending of steering catheter 600. Accordingly, the rotational alignment of outer sheath 500 with steering catheter 600 is beneficial to the proper positioning of valve cover 550 and prosthetic mitral valve 100 relative to mitral valve annulus 158. As an alternative to the engagement of luer lock 532 within slot 142, outer sheath 500 may be rotationally aligned with and fixed relative to steering catheter 600 using any other key and corresponding keyway feature, slots and corresponding tabs, or other rotational keying mechanisms known in the art. Alternatively or additionally, alignment markers may be provided at the proximal end of catheter assembly 400 to visually indicate rotational alignment.

Extension catheter 700 extends proximally through steering catheter 600 and steering catheter handle 132 where its proximal end 702 may be fixedly coupled to an extension catheter holder 730 positioned proximally of the steering catheter handle. Extension catheter holder 730 may be in the form of a disk having a central aperture (not shown) that enables suture catheter 800 to extend proximally therethrough. A flush port (not shown) may be positioned on the rim of extension catheter holder 730 to allow flushing of the interior of extension catheter 700.

Suture catheter 800 extends proximally through extension catheter 700, steering catheter handle 132, and extension catheter holder 730 and may be fixedly coupled at its proximal end 804 to a suture catheter control 870 positioned proximally of the extension catheter holder. Suture catheter control 870 may be in the form of a disk or puck having a central aperture (not shown) through which nosecone catheter 900 may extend proximally. A flush port (not shown) may be positioned on the rim of suture catheter control 870 to allow flushing of the interior of suture catheter 800.

Nosecone catheter 900 extends proximally from nosecone 950 through suture catheter 800, steering catheter handle 132, extension catheter holder 730 and suture catheter control 870 and may be coupled to a nosecone catheter control 920. Nosecone catheter control 920 may be in the form of a disk or puck to which a proximal portion of nosecone catheter 900 may be fixedly attached. A central aperture (not shown) through nosecone catheter control 920 enables the proximal end 902 of nosecone catheter 900 and guidewire 975 to extend proximally of the nosecone catheter control. Although both suture catheter control 870 and nosecone catheter control 920 have been shown and described as disks or pucks, they may have different shapes, including spheres, ovoids, polygons or other shapes that may be grasped and translated. In addition, the shape of suture catheter control 870 may be the same as or different than the shape of nosecone catheter control 920.

Both suture catheter control 870 and nosecone catheter control 920 may be locked in longitudinal positions relative to extension catheter holder 730 or may be selectively released so that the suture catheter control is translatable proximally and distally relative to the extension catheter holder and the nosecone catheter control, and the nosecone catheter control is translatable proximally and distally relative to the extension catheter holder and the suture catheter control. In order to maintain the axial alignment of suture catheter control 870 and nosecone catheter control 920 with extension catheter holder 730 (and also the axial alignment of the components of catheter assembly 400), both the suture catheter control and the nosecone catheter control may translate along a plurality of rigid alignment rods 995. Each alignment rod 995 may be connected at its distal end to extension catheter holder 730 and may extend proximally therefrom through an aperture in suture catheter control 870 and another aperture in nosecone catheter control 920. An enlarged fitting 997 may be positioned on the free end of each alignment rod 995 to prevent nosecone catheter control 920 and suture catheter control 870 from being removed from the rods. Alignment rods 995 assure that suture catheter control 870 and nosecone catheter control 920 freely slide in the proximal and distal directions as handle assembly 300 is operated.

In some embodiments, suture catheter control 870 and/or nosecone catheter control 920 may include a release mechanism 880 that interacts with a threaded rod or lead screw 999 fixedly connected at its distal end to extension catheter holder 730 and extending proximally therefrom through the suture catheter control and nosecone catheter control. Release mechanism 880 may be biased to engage threaded rod 999, preventing suture catheter control 870 from translating proximally or distally relative to extension catheter holder 730 and nosecone catheter control 920, and preventing the nosecone catheter control from translating proximally or distally relative to the extension catheter holder and the suture catheter control. Release mechanism 880 may incorporate a spring-loaded pin, toggle release, snap shackle, quick-release skewer, set screw or another device to provide effective and rapid release from threaded rod 999. Actuating one or more of release mechanisms 880 will selectively free either suture catheter control 870 or nosecone catheter control 920, or both, to translate proximally or distally to desired positions. Moving suture catheter control 870 proximally or distally relative to nosecone catheter control 920 will selectively translate suture catheter 800 relative to nosecone catheter 900, nosecone 950 and the other components of catheter assembly 400. Moving nosecone catheter control 920 proximally or distally relative to suture catheter control 870 will selectively translate nosecone catheter 900 and nosecone 950 relative to suture catheter 800 and the other components of catheter assembly 400. Although the use of a threaded rod 999 is shown, handle assembly 300 may include a rod having other structures (grooves, divots, depressions, etc.) for engaging with release mechanisms 880 when in the engaged position.

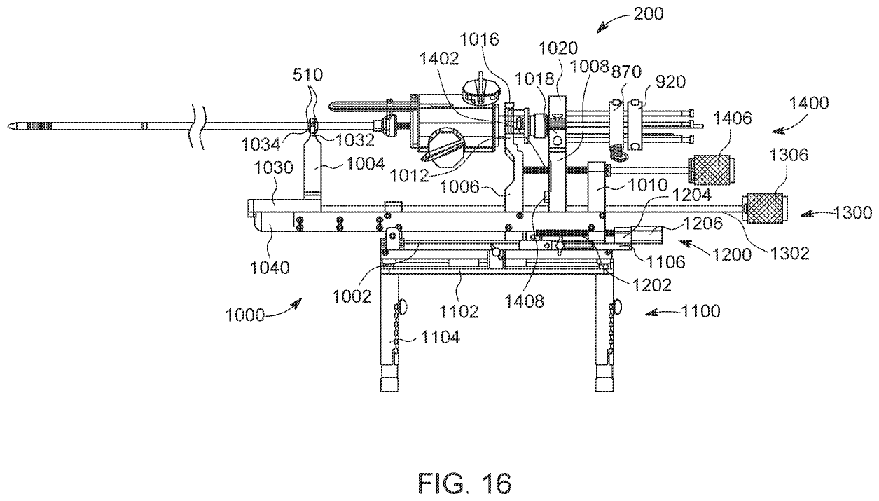

Referring to FIG. 16, delivery system 200 may be supported by a stabilizer 1000 that includes a base 1002 and a plurality of rigid supports 1004, 1006, 1008 and 1010. A handle support 1006 may be fixedly connected to base 1002 and may extend upward to a semi-circular cradle 1012 that supports a disk-shaped member 1014 (FIG. 15) connected to the proximal end of steering catheter handle 132. A cover 1016 may be assembled to cradle 1012 over disk-shaped member 1014 to secure the disk-shaped member and steering catheter handle 132 to handle support 1006. Cradle 1012 and cover 1016 together support disk-shaped member 1014 in a manner that enables steering catheter handle 132 to be rotated about its longitudinal axis in both clockwise and counterclockwise directions. Support 1010 also may be fixedly connected to base 1002 and may extend upward therefrom to a height lower than that of handle support 1006. An extension catheter support 1008 between handle support 1006 and support 1010, however, may be spaced above and translatable relative to base 1002. Extension catheter support 1008 may extend upward to a semi-circular cradle 1018 having a recess sized and shaped to receive extension catheter holder 730. A cover 1020 may be assembled to cradle 1018 over extension catheter holder 730 to secure the extension catheter holder in its assembled position.

Outer sheath support 1004 may be fixedly mounted to a slider block 1030 near the distal end of base 1002 and may extend upward to a semi-circular cradle 1032 sized to receive outer sheath 500. Cradle 1032 has a width sized to be received between two rings 510 spaced from one another around the exterior of outer sheath 500. The engagement of cradle 1032 between rings 510 prevents outer sheath 500 from translating proximally or distally relative to outer sheath support 1004. A cover 1034 may be assembled to outer sheath support 1004 over outer sheath 500 to secure the outer sheath in its assembled position. Slider block 1030 may be positioned within a channel in the top surface of a slider block guide 1040. Slider block 1030 may slide in slider block guide 1040 distally and proximally relative to base 1002 and delivery system 200 mounted thereon.

Stabilizer 1000 may be slidably mounted to a portable table 1100 that includes a platform 1102 supported by a plurality of height-adjustable legs 1104. A second platform 1106 consisting of two plates pivotally connected at one end may be mounted between platform 1102 and the base 1002 of stabilizer 1000 in a manner that enables the base to slide proximally and distally relative to table 1100 and that enables the proximal end of the base to be raised or lowered relative to platform 1102, orienting delivery system 200 at a transverse angle to horizontal.

Stabilizer 1000 also includes a plurality of actuators that selectively control the translation of base 1002 and the translation of the components of catheter assembly 400 relative to one another. Base actuator 1200 may include a threaded rod or lead screw 1202 that is engaged with internal threads in support 1010 and that also may be coupled to a support post 1204 fixedly attached to second platform 1106. The coupling of lead screw 1202 to support post 1204 is such that the lead screw is rotatable but not translatable relative to support post 1204. Rotating lead screw 1202 in one direction (via an actuating knob 1206) will cause base 1002 and all of the components assembled to the base to advance distally relative to second platform 1106 and table 1100, while rotating the lead screw in the opposite direction will cause the base and the components assembled to the base to retract proximally relative to the second platform and the table.

An outer sheath actuator 1300 includes a rod 1302, at least a distal portion of which is threaded, supported for rotation through handle support 1006, extension catheter support 1008 and support 1010. The distal end of threaded rod or lead screw 1302 is journaled for rotation in slider block 1030, and an actuating knob 1306 may be affixed to its proximal end. Lead screw 1302 rotates freely within apertures in handle support 1006 and support 1010, while being engaged with threads within extension catheter support 1008 so that the lead screw rotates but does not translate relative supports 1006 and 1010, but translates relative to support 1008. As a result, while extension catheter support 1008 is held in a fixed position, rotation of lead screw 1302 in one direction causes slider block 1030 to translate proximally, retracting outer sheath 500 relative to the other components of catheter assembly 400. Rotation of lead screw 1302 in the opposite direction causes slider block 1030 to translate distally, advancing outer sheath 500 relative to the other components of catheter assembly 400. A pair of smooth, rigid rods (not shown) may extend from slider block 1030 proximally through apertures in handle support 1006, extension catheter support 1008 and support 1010 to maintain the alignment of outer sheath support 1004 with the other portions of delivery system 200 as they translate relative to one another. The retraction of outer sheath 500 may be utilized to deploy a prosthetic heart valve held within valve cover 550 at the distal end of the outer sheath, while the advancement of the outer sheath/valve cover over the prosthetic heart valve may potentially be utilized to retract the valve back into the valve cover if it hasn't been fully deployed. Arrangements for retrieving a more fully deployed prosthetic heart valve are described below.

Stabilizer 1000 may also include a valve positioning actuator 1400 that translates several of the components of catheter assembly 400 together relative to steering catheter 600. Positioning actuator 1400 includes a rod 1402, at least a distal portion of which is threaded. Threaded rod or lead screw 1402 is supported for rotation at its distal end in handle support 1006, extends through extension catheter support 1008, and is supported for rotation at its proximal end in support 1010. Positioning actuator 1400 is journaled in handle support 1006 and support 1010 so that it does not translate relative to those supports as it is rotated. However, lead screw 1402 is threadedly engaged with internal threads in extension catheter support 1008. As noted previously, extension catheter holder 730 is captured in the cradle 1018 in extension catheter support 1008, preventing the extension catheter holder from translating proximally or distally relative to the extension catheter support. Similarly, steering catheter handle 132 is supported by handle support 1006, which is held in a fixed position on base 1002. As a result of this arrangement, rotation of positioning actuator 1400 (via actuating knob 1406) results in the translation of extension catheter support 1008 relative to handle support 1006 and support 1010. The translation of extension catheter support 1008 results in the simultaneous translation of suture catheter control 870 and nosecone catheter control 920 by virtue of their connection to threaded rod 999, as well as the simultaneous translation of outer sheath 500 by virtue of its connection to lead screw 1302. A release mechanism 1408 may be incorporated on extension catheter support 1008 to release the extension catheter support from lead screw 1402, enabling the rapid manual translation of the extension catheter support either proximally or distally, thereby translating extension catheter holder 730, suture catheter control 870 and nosecone catheter control 920 simultaneously to either advance prosthetic mitral valve 100 relative to mitral valve annulus 158 or to retract it.

In view of the forgoing connections among the various components, the rotation of positioning actuator 1400 in a first direction to translate extension catheter support 1008 simultaneously causes slider block 1030, extension catheter holder 730, suture catheter control 870 and nosecone catheter control 920 to retract proximally, while the position of steering catheter handle 132 remains fixed. Rotation of positioning actuator 1400 in the opposite direction causes the advancement in the distal direction of slider block 1030, extension catheter holder 730, suture catheter control 870 and nosecone catheter control 920, again without translating steering catheter handle 132.

As explained above, suture catheter control 870 may be translated proximally or distally relative to the other components of catheter assembly 400 by depressing release mechanism 880 and manually sliding the suture catheter control proximally or distally. Translation of suture catheter control 870 causes suture catheter 800 to translate relative to each of outer sheath 500, steering catheter 600, extension catheter 700, nosecone catheter 900 and nosecone 950. As will be explained below, the proximal retraction of suture catheter 800 relative to extension catheter 700 and/or outer sheath 500 increases the axial tension on tethers 850 to keep them connected to prosthetic mitral valve 100 and to maintain the prosthetic valve in a pre-deployed position within delivery system 200, while the distal advancement of the suture catheter relative to the extension catheter and/or the outer sheath releases the axial tension on the tethers and allows deployment of the prosthetic mitral valve.

Prosthetic mitral valve 100 may be loaded into valve cover 550 by first removing nosecone 950 from nosecone catheter 900 and flushing all of the components of catheter assembly 400 to remove air. Distal suture ring 830 with prosthetic mitral valve 100 attached to it through tethers 850 may then be assembled to proximal suture ring 820 at the distal end of suture catheter 800, and the suture catheter may be retracted to collapse the prosthetic mitral valve and draw it toward valve cover 550. A loading funnel removably attached to the distal end of valve cover 550 may assist in collapsing the valve to facilitate the loading process. Suture catheter 800 may be retracted until the tips of the atrial petals 114 of the prosthetic valve are seated in the can 720 at the distal end of extension catheter 700. Outer sheath 500 and valve cover 550 may then be advanced until prosthetic mitral valve 100 is fully positioned within the valve cover. Nosecone 950 may then be assembled to the distal end of nosecone catheter 900 and the nosecone catheter may be withdrawn until the nosecone seats against the distal end of valve cover 550. In a preferred arrangement, suture catheter 800 and tethers 850 are pretensioned after prosthetic mitral valve 100 has been loaded into valve cover 550 and prior to the insertion of catheter assembly 400 into the patient. To pretension suture catheter 800 and tethers 850, a torque wrench can be assembled to the proximal end of threaded rod 999 and the rod can be turned to retract the suture catheter relative to extension catheter 700 until a desired tension is achieved. More particularly, once the can 720 at the distal end of extension catheter 700 is seated against the tip ring 620 of steering catheter 600, any further tension applied to suture catheter 800 will be transferred to the steering catheter. Applying further tension to suture catheter 800 (through further rotation of threaded rod 999) will cause the suture catheter to lengthen slightly by the separation of the laser cuts at its distal end, creating a spring-like tension in the suture catheter. The desired tension should be within a range that is sufficient to hold prosthetic mitral valve 100 securely within can 720 at the distal end of extension catheter 700 and to maintain the connection of tethers 850 to the pins 118 on the prosthetic valve, but not so high as to significantly decrease the flexibility of the distal end of catheter assembly 400. The tension/compression relationship between suture catheter 800 and extension catheter 700 should be maintained until the atrial petals 114 of prosthetic mitral valve 100 have been released from extension catheter can 720. Delivery system 200 is now ready for delivering prosthetic mitral valve 100 into the left atrium LA of the patient and deploying the prosthetic valve in mitral valve annulus 158. Methods for preparing delivery system 200 and for loading prosthetic mitral valve 100 therein are described in more detail in U.S. Provisional Application No. 63/382,012, filed Nov. 2, 2022, the disclosure of which is hereby incorporated by reference herein.

In a conventional procedure, an incision is made in the patient's groin to access the femoral vein, and a guidewire, such as guidewire 975, is fed up through the femoral vein, advanced through the inferior vena cava 150 to the right atrium RA, through a puncture in intra-atrial septum 154 and into the left atrium LA. Catheter assembly 400 is then advanced over guidewire 975 until nosecone 950 is located in left ventricle LV and valve cover 550 is positioned in mitral valve annulus 158 such that a distal portion of prosthetic mitral valve 100 is located on the ventricular side of the annulus and a proximal portion of the prosthetic mitral valve is located on the atrial side of the annulus. Outer sheath 500 and valve cover 550 may then be partially retracted while nosecone 950 is held in place, thereby creating a separation between the nosecone and the valve cover. As valve cover 550 is retracted, it exposes the ventricular anchor 108 of prosthetic mitral valve 100, causing it to expand within left ventricle LV.

Following the release and expansion of ventricular anchor 108, outer sheath 500 and valve cover 550 may be further retracted to fully expose prosthetic mitral valve 100 (i.e., atrial anchor 106), while maintaining tension in tethers 850. Once fully exposed, prosthetic mitral valve 100 may be retracted proximally to bring the ventricular anchor into contact against the ventricular side of mitral valve annulus 158. This may be accomplished by rotating positioning actuator 1400 to retract extension catheter 700 and suture catheter 800. If extension catheter 700 is already in a fully retracted position, ventricular anchor 108 may be brought into contact with the ventricular side of mitral valve annulus 158 by rotating prosthetic mitral valve 100. This may be done by rotating steering catheter handle 132 in the posterior direction. The rotation of steering catheter handle 132 will cause the simultaneous rotation of outer sheath 500 and valve cover 550 by virtue of the engagement of the luer lock 532 of hemostasis valve 530 within the slot 142 in rigid keyway 140. As a result of the bends at the distal end of catheter assembly 400, rotating steering catheter handle 132 and valve cover 550 will cause prosthetic mitral valve 100 to pivot relative to the plane of mitral valve annulus 158 until ventricular anchor 108 contacts the mitral valve annulus. Ventricular anchor 108 will deflect as it contacts mitral valve annulus 158, enabling prosthetic mitral valve 100 to move closer to its proper orientation relative to the valve annulus, with atrial anchor 106 on the atrial side of mitral valve annulus 158.

At this point, prosthetic mitral valve 100 may still be held by tethers 850 in a not yet fully deployed condition, with atrial anchor 106 still held in a collapsed condition within the can 720 of extension catheter 700. Since leaflets 113 are not yet functioning when ventricular anchor 108 is retracted against mitral valve annulus 158, the positioning of the ventricular anchor will impede the flow of blood from left atrium LA to left ventricle LV. Therefore, as ventricular anchor 108 is retracted against mitral valve annulus 158, it is desirable to simultaneously advance suture catheter 800 to release atrial anchor 106 so that blood can begin flowing through the prosthetic valve. This may be accomplished by actuating release mechanism 1408 to quickly retract extension catheter support 1008 and extension catheter 700, drawing ventricular anchor 108 against the mitral valve annulus and, at the same time, actuating release mechanism 880 to translate suture catheter control 870 and suture catheter 800 distally to relieve the tension in tethers 850, enabling atrial anchor 106 to expand and release from can 720. Upon its expansion, atrial anchor 106 will contact the atrial side of mitral valve annulus 158, thus sandwiching the mitral valve annulus between the atrial anchor and the ventricular anchor and securing prosthetic mitral valve 100 in place. After prosthetic mitral valve 100 has been detached from suture catheter 800, the suture catheter may be retracted, pulling tethers 850 back into extension catheter 700. Nosecone catheter 900 may then be retracted proximally until nosecone 950 is seated against the distal end of valve cover 550, and catheter assembly 400 may be removed from the patient.

As noted, it may become necessary during the deployment of prosthetic mitral valve 100 to recapture the prosthetic valve after atrial anchor 106 has been deployed from valve cover 550, but while the prosthetic valve is still retained by tethers 850. As noted previously, outer sheath 500 is retracted to deploy prosthetic mitral valve 100. During the retraction of outer sheath 500, internal friction between the collapsed mitral valve and valve cover 550 will inhibit the retraction of the coiled layer 514 at the distal end of the outer sheath, causing the individual coils to separate farther from one another and braided sleeve 516 to lengthen and collapse around the coiled layer. The structure of the coil will prevent the braid from lengthening and collapsing further, and the braid will lock onto the coil like a Chinese finger trap. The radial strength of the coil prevents coil/braid portion 514/516 from ovalizing, enabling the coil/braid to be pulled back over the deflected shape of steering catheter 600, even when high tension forces are applied to deploy prosthetic mitral valve 100.

In order to recapture prosthetic mitral valve 100, however, very high compression forces on the outer layer are required to push the valve cover distally. Since the prosthetic mitral valve is at body temperature, which is above the AF temperature of the nitinol forming the valve frame 102 (which should be below body temperature and is typically set to around 20° C.), the frame is more rigid (and more difficult to collapse) than it is at room temperature when initially loaded into valve cover 550. Also, there is no funnel available to assist in collapsing the prosthetic valve into valve cover 550. As a result, to recapture prosthetic mitral valve 100, compression forces of about 50 lbs. to about 150 lbs. or more are required at the distal end of catheter assembly 400. However, the large gaps created between adjacent turns of coiled layer 514 during the deployment of prosthetic mitral valve 100 make it impossible, or at least very difficult, to generate such compressive forces at the distal end of the catheter assembly. As the prosthetic valve is retracted against valve cover 550, any compressive forces created will simply cause the individual coils of coiled layer 514 to collapse against one another, such that the coil/braid portion 514/516 will foreshorten by 50% or more. Since braided sleeve 516 is fixedly attached at both ends to coiled layer 514, it also will have to shorten by the same amount, which either may be impossible or will cause the braided sleeve to increase in diameter to a size that makes it difficult to remove catheter assembly 400 from the patient.

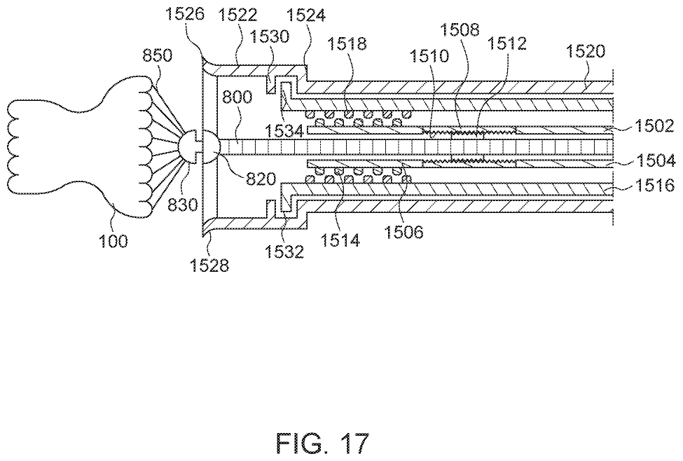

One approach to overcoming the drawbacks of delivery system 200 when it is desired to recapture a partially deployed prosthetic valve is to employ a special retrieval catheter, such as retrieval catheter 1500, the distal end of which is shown in FIG. 17. Although the following will describe the use of retrieval catheter 1500 to recapture prosthetic mitral valve 100, the retrieval catheter is not so limited and may be used to recapture other partially deployed cardiac valves or other partially deployed medical devices that need to be re-collapsed for removal from a patient. Retrieval catheter 1500 includes an inner shaft 1502 formed from a proximal portion 1504, a distal portion 1506, and an intermediate portion 1508 laser welded between portions 1504 and 1506. Portions 1504 and 1506 may be lengths of a torque shaft or tri-coil structure (such as the one in the distal section 708 of extension catheter 700 described above) having sufficient flexibility to enable retrieval catheter 1500 to follow the deflected shape of suture catheter 800, while still being able to withstand compression forces and transmit torque. Intermediate portion 1508 may be formed from a length of stainless-steel hypotube and includes a series of threads 1510 on its inner diameter. Threads 1510 are intended to mate with a threaded portion 1512 laser welded or otherwise fixedly secured to the exterior of suture catheter 800. In that regard, threaded portion 1512 may have an outer diameter that is about equal to the outer diameter of proximal suture ring 820 and distal suture ring 830, and threads 1510 may have a very slightly larger inner diameter. A helical stainless-steel coil 1514, the purpose of which will be described below, is longitudinally stretched to separate the turns of the coil and laser welded or otherwise fixedly connected to the outer diameter of distal portion 1506.

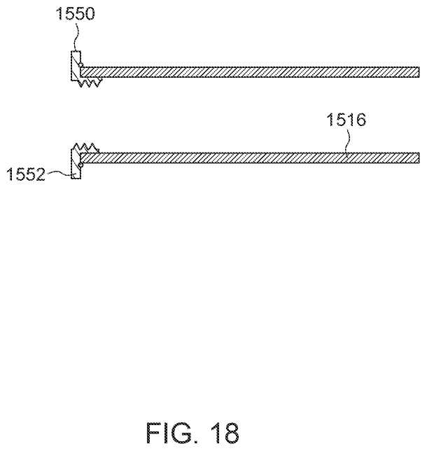

A further shaft 1516 is fitted over shaft 1502. Shaft 1516 may be formed from a laser cut hypotube or another structure that is somewhat flexible yet still able to transmit torque, such as, for example, a tri-coil structure or a braided polymeric shaft. At its distal end, the inner diameter of shaft 1516 may include a length of a helical stainless-steel coil 1518 that has been longitudinally stretched to mate with coil 1514. The mating of coil 1518 with coil 1514 acts as a screw engagement such that rotation of shaft 1516 relative to inner shaft 1502 will cause shaft 1516 to translate proximally or distally relative to inner shaft 1502, depending on the direction in which shaft 1516 is rotated. Although lengths of threaded hypotube can perform the same function as mating coils 1514 and 1518, the use of helical coils enables at least the distal end of retrieval catheter 1500 to be flexible so that it can follow the curvature of suture catheter 800 as it is advanced toward expanded mitral valve 100. Coil 1518 may be relatively short (only one or a few turns) as long as coil 1514 has a sufficiently long length to recapture the prosthetic valve. Rather than an internal coil 1518, a flange 1550 may be laser welded at one end to the inner diameter at the distal end of shaft 1516, as shown in FIG. 18. Flange 1550 may be internally threaded and may have an outwardly projecting ring 1552 at its distal end, which will reside in the annular pocket 1532 in a valve cover 1522 described below. As noted, flange 1550 may have a relatively short length of internal threads that mate with a relatively longer coil 1514 on inner shaft 1502. In a still further arrangement, shaft 1516 need not have either an inner coil or an inner threaded portion. Rather, a single continuous ring or series of partial ring sections (not shown) may be laser welded to the inner diameter of shaft 1516. The ring or ring sections can be sized and shaped to follow the turns of coil 1514 as shaft 1516 is rotated.

Retrieval catheter 1500 further includes an outer shaft 1520 that is assembled over shaft 1516. Outer shaft 1520 may be formed from a laser cut hypotube, tri-coil, coil/braid structure or other flexible tubular structure so as to maintain the flexibility of retrieval catheter 1500. At its distal end, outer shaft 1520 includes a valve cover 1522 that has a proximal end 1524, a distal end 1526, and an inner diameter and length that are similar to those of valve cover 550 so as to be able to receive prosthetic mitral valve 100 in a collapsed condition. The distal end 1526 of valve cover 1522 may have an outwardly flared portion 1528 sized to receive the expanded prosthetic valve and facilitate its collapse as it enters the valve cover. Alternatively or additionally, the distal end 1526 of valve cover 1522 may have a rounded edge so as to not damage or minimize damage to prosthetic heart valve 100 as it collapses and enters the valve cover. An annular ring 1530 on the inside of valve cover 1522 defines an annular pocket 1532 at the proximal end 1524 of the valve cover. Pocket 1532 is sized to receive an annular flange 1534 at the distal end of shaft 1516. Flange 1534 is captured in pocket 1532 so that shaft 1516 is able to rotate but not translate relative to outer shaft 1520.

Should it become necessary to retrieve an expanded prosthetic heart valve following deployment from delivery system 200, the following procedure may be followed to employ retrieval catheter 1500. Although the following description assumes that delivery system 200 was used to deliver prosthetic mitral valve 100 to the target site and deploy it, it will be appreciated that retrieval catheter 1500 can be used in conjunction with any delivery system that continues to hold the prosthetic heart valve in place while allowing components of the delivery system to be removed and the retrieval catheter to be assembled to the remaining delivery system components. The following description also assumes that delivery system 200 includes a nosecone catheter 900 which remains in place within suture catheter 800 during the retrieval procedure. It will be appreciated, however, that the delivery system may not include such a nosecone catheter.

As a first step, suture catheter 800 is disconnected from handle assembly 300 and stabilizer 1000. That is, the proximal end 804 of suture catheter 800 is uncoupled from the insert in suture catheter control 870. An elongated extension tube or rod (not shown) having an outer diameter that is substantially similar to that of suture catheter 800 may then be coupled to the proximal end 804 of the suture catheter. Similarly, another elongated extension tube or rod may be coupled to the proximal end of nosecone catheter 900. With the extension tubes and/or rods in place, the outer components of catheter assembly 400 (i.e., the components that surround suture catheter 800) may be removed. More particularly, outer sheath 500 (with valve cover 550), steering catheter 600 and extension catheter 700 may be uncoupled from their respective controls in handle assembly 300 and translated proximally over the extension tubes and/or rods either individually or collectively. The additional length of the extension tubes and/or rods enables the positions of suture catheter 800 and nosecone catheter 900 to be controlled as these other components are withdrawn. Nosecone catheter 900 may remain in place within suture catheter 800 with nosecone 950 at its distal end.

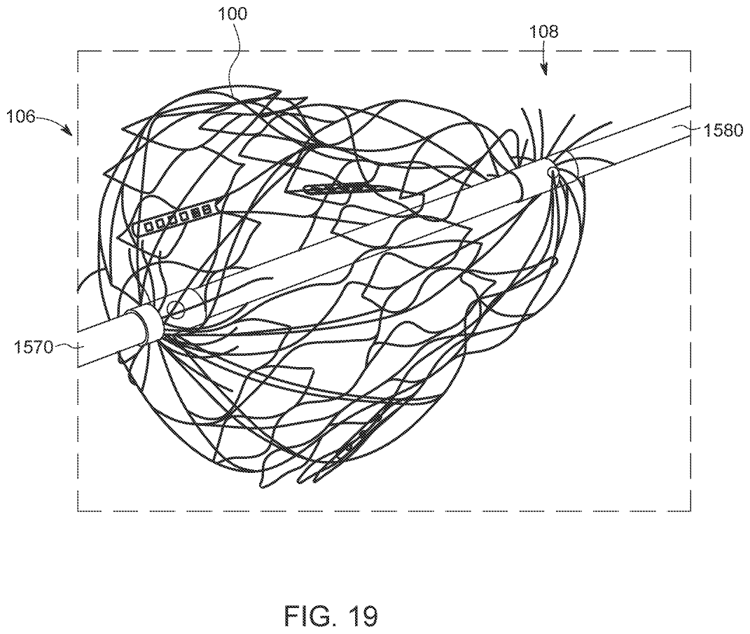

At this point it should be noted that, in order to retrieve prosthetic mitral valve 100, it is important that tethers 850 remain connected to the prosthetic valve, even when the tethers are no longer under tension. Although methods for maintaining the connection of tethers 850 to prosthetic mitral valve 100 are beyond the scope of this disclosure, two possible methods may be briefly mentioned. In one method, the pins 118 on prosthetic mitral valve 100 may each be formed with an aperture through which the ends of tethers 850 may be threaded. A separate suture may then be threaded through the loops at the ends of the tethers. This suture may have one end fixedly connected to an elongated rod that may be manipulated by the user, and a loop at the other end that is assembled around the rod. When it is desired to release tethers 850 from prosthetic mitral valve 100, the user may pull the rod proximally to release the looped end of the suture from the rod and then remove the suture from the looped ends of the tethers. Tethers 850 are then free to be pulled from the apertures in pins 118, fully releasing the prosthetic valve. Another method eliminates proximal suture ring 820 and distal suture ring 830 and instead connects the prosthetic heart valve to a pair of telescopically connected tubes that may be rotated in opposite directions relative to one another by a control device to either collapse or expand the prosthetic valve. Referring to FIG. 19, atrial anchor 106 is connected by a first series of tethers to a first tube 1570 and ventricular anchor 108 is connected by a second series of tethers to a second tube 1580. Tubes 1570 and 1580 telescopically engage one another and can be rotated in opposite directions relative to one another. When tube 1570 is rotated in a first direction and tube 1580 is rotated in a second opposite direction, the first series of tethers will wind around tube 1570, collapsing atrial anchor 106 radially inward, and the second series of tethers will wind around tube 1580, collapsing ventricular anchor 108 radially inward. Rotating tubes 1570 and 1580 in the opposite directions, that is, rotating tube 1570 in the second direction and tube 1580 in the first direction, will release the tension in the tethers, enabling mitral valve 100 to expand (if unconstrained by a valve cover or other structure). The tethers may be released from the atrial and ventricular anchors by continued rotation of tube 1570 in the second direction and tube 1580 in the first direction. The control device, method of operating same, and method of disconnecting same from the prosthetic heart valve are described in PCT Patent Publication No. WO 2022/029111, the entire disclosure of which is hereby incorporated by reference herein. Other methods for maintaining the connection of tethers 850 to prosthetic mitral valve 100 in a releasable fashion are contemplated herein and will be known to those skilled in the art.

Following the removal of outer sheath 500, steering catheter 600 and extension catheter 700, retrieval catheter 1500 may be advanced over the extension tubes and/or rods, nosecone catheter 900 and suture catheter 800 to a position just proximal of prosthetic mitral valve 100. During the advancement of retrieval catheter 1500, suture catheter 800, nosecone catheter 900 and the extension tubes and/or rods act as a strong rail to guide the retrieval catheter to the desired location, thereby avoiding the need for a separate guidewire. Retrieval catheter 1500 is advanced to a position at which the threads 1510 in intermediate portion 1508 first contact the threaded portion 1512 on suture catheter 800. At that point, retrieval catheter 1500, and particularly inner shaft 1502, may be rotated about its longitudinal axis to engage threads 1510 with threaded portion 1512, thereby locking the inner shaft to suture catheter 800 so they are not able to translate longitudinally relative to one another. Subsequently, shaft 1516 may be rotated about its longitudinal axis while inner shaft 1502 remains stationary. This rotation will cause coil 1518 to travel in a screw-like fashion along coil 1514, either advancing shaft 1516 distally or retracting shaft 1516 proximally relative to inner shaft 1502 and suture catheter 800, depending on the direction of rotation. A keying mechanism at or near the proximal end of outer shaft 1520 may prevent the outer shaft from rotating as shaft 1516 is rotated. Since the flange 1534 at the distal end of shaft 1516 is captured in the pocket 1532 in valve cover 1522, the translation of shaft 1516 will result in a simultaneous translation of outer shaft 1520 and valve cover 1522 relative to suture catheter 800 and prosthetic mitral valve 100.

To retrieve prosthetic mitral valve 100, shaft 1516 is rotated to advance outer shaft 1520 and valve cover 1522 distally toward the prosthetic valve. As valve cover 1522 contacts prosthetic mitral valve 100, flared portion 1528 will begin to collapse the prosthetic valve and guide it into the valve cover. Prosthetic mitral valve 100 will remain stationary as a result of the tension in tethers 850 and the fact that suture catheter 800 is held in a fixed position. Shaft 1516 continues to be rotated, collapsing prosthetic mitral valve 100 as valve cover 1522 is advanced distally over it, until the prosthetic valve is housed completely within the valve cover. Although the forces required to advance valve cover 1522 over prosthetic mitral valve 100 are quite substantial, they are generated near the distal end of retrieval catheter 1500, much closer to where they are needed. As a result, these forces are much more effective than forces generated at or near the proximal end of suture catheter 800, the effects of which forces would be significantly dampened as they attempt to compress the length of outer sheath 500. Once prosthetic mitral valve 100 has been fully captured within valve cover 1522, nosecone catheter 900 can be retracted proximally until nosecone 950 is positioned against the distal end of the valve cover, and the entire assembly, including retrieval catheter 1500, suture catheter 800, nosecone catheter 900 and prosthetic mitral valve 100, can be removed from the patient.

The retrieval procedure described above requires very little deflection of the distal end of the catheter assembly. However, if prosthetic mitral valve 100 is positioned in mitral valve annulus 158, the catheter assembly will be substantially deflected. Even if retrieval catheter 1500 is able to generate the forces required to re-sheathe the prosthetic mitral valve, the damage to the heart tissue will be significant. Therefore, in such situations, it would be preferable to reduce the size of prosthetic mitral valve 100 so that it is more easily removed from mitral valve annulus 158. This may be achieved by using the device shown in FIG. 19 and described above. By rotating tubes 1570 and 1580 in opposite directions, tethers 850 will be placed under tension, collapsing atrial anchor 106 and ventricular anchor 108. This will enable the prosthetic mitral valve to be axially aligned with retrieval catheter 1500 and to follow the retrieval catheter axis as it enters valve cover 1522.

To summarize the foregoing, the present disclosure describes a retrieval catheter for retrieving a partially deployed medical device held by tethers or otherwise at the distal end of a delivery catheter. The retrieval catheter includes an inner tubular shaft engageable with the delivery catheter in a manner that prevents translation of the delivery catheter in a longitudinal direction relative to the inner tubular shaft absent rotation of the delivery catheter relative to the inner tubular shaft, the inner tubular shaft having a proximal end, a distal end, and a lumen extending therethrough from the proximal end to the distal end, an outer surface in a distal portion of the inner tubular shaft having a series of outer threads. The retrieval catheter also includes an intermediate tubular shaft assembled around the inner tubular shaft, the intermediate tubular shaft having a proximal end, a distal end, and an inner surface defining a lumen therethrough from the proximal end to the distal end, the inner surface in a distal portion of the intermediate tubular shaft having a series of inner threads engageable with the outer threads of the inner shaft such that rotation of the intermediate tubular shaft relative to the inner tubular shaft translates the intermediate tubular shaft proximally or distally relative to the inner tubular shaft and the delivery catheter. An outer tubular shaft is assembled around the intermediate tubular shaft, the outer tubular shaft having a proximal end, a distal end, and a lumen therethrough from the proximal end to the distal end. A valve cover at the distal end of the outer tubular shaft is operatively connected to the intermediate tubular shaft such that translation of the intermediate tubular shaft translates the valve cover; and/or
the distal portion of the inner tubular shaft may be flexible; and/or
the distal portion of the inner tubular shaft may include a flexible distal section, a flexible proximal section, and a rigid tubular portion connected between the distal section and the proximal section; and/or
an inner surface of the rigid tubular portion may include a first plurality of threads; and/or
an outer surface of the delivery catheter may include a section having a second plurality of threads that are complementary to the first plurality of threads, whereby the engagement of the second plurality of threads with the first plurality of threads prevents the translation of the delivery catheter in the longitudinal direction relative to the inner tubular shaft absent rotation of the delivery catheter relative to the inner tubular shaft; and/or
the intermediate tubular shaft may be flexible; and/or
the intermediate tubular shaft may have a tri-coil structure and/or
the intermediate tubular shaft may be formed from a hypotube having a plurality of circumferential slits; and/or
the outer tubular shaft may be flexible; and/or
the outer tubular shaft may have a tri-coil structure; and/or
the outer tubular shaft may be formed from a hypotube having a plurality of circumferential slits; and/or
the series of outer threads may include a plurality of turns of a helical coil fixedly connected to the outer surface in the distal portion of the inner tubular shaft; and/or
the series of inner threads may include a plurality of turns of a helical coil fixedly connected to the inner surface in the distal portion of the intermediate tubular shaft; and/or
the valve cover may have a proximal end connected to the outer tubular shaft, an open distal end and an annular ring positioned inside the valve cover so as to define an annular pocket between the annular ring and the proximal end of the valve cover; and/or
the distal end of the intermediate tubular shaft may include an annular flange, the annular flange being captured within the annular pocket in the valve cover so that the intermediate tubular shaft is rotatable relative to the outer tubular shaft and not translatable relative to the outer tubular shaft; and/or
the valve cover may have a proximal end connected to the outer tubular shaft and an open distal end, the valve cover at the open distal end flaring outwardly; and/or
the valve cover may have a proximal end connected to the outer tubular shaft and an open distal end, the open distal end having a rounded edge.

The present disclosure also describes a method for retrieving a partially deployed medical device held at a distal end of a catheter assembly within a patient, the catheter assembly including an outer sheath having a proximal end and a distal end, a first valve cover at the distal end of the outer sheath, a steering catheter disposed within the outer sheath, an extension catheter disposed within the steering catheter, and a suture catheter disposed within the extension catheter, the medical device being positioned outside of the valve cover and connected to the suture catheter by a plurality of tethers. The method includes removing the extension catheter, the steering catheter and the outer sheath including the valve cover from the catheter assembly and from the patient, and assembling a retrieval catheter over the suture catheter within the patient. The retrieval catheter includes an inner tubular shaft engageable with the suture catheter in a manner that prevents translation of the suture catheter in a longitudinal direction relative to the inner tubular shaft; an intermediate tubular shaft assembled around the inner tubular shaft, the intermediate tubular shaft being threadedly engaged with the inner tubular shaft; an outer tubular shaft assembled around the intermediate tubular shaft; and a second valve cover at the distal end of the outer tubular shaft, the second valve cover being operatively connected to the intermediate tubular shaft. The method further includes rotating the intermediate tubular shaft relative to the inner tubular shaft to translate the intermediate tubular shaft and the second valve cover distally relative to the suture catheter and the medical device until the second valve cover collapses and covers the medical device; and removing the retrieval catheter, the suture catheter, and the medical device from the patient; and/or
the step of assembling the retrieval catheter over the suture catheter may include threadedly engaging the inner tubular shaft to the suture catheter; and/or
the catheter assembly may further include a nosecone catheter extending through the suture catheter and the medical device, and a nosecone on a distal end of the nosecone catheter, the nosecone being positioned distally of the medical device, and the method may further include, after the second valve cover covers the medical device and before the removing step, retracting the nosecone catheter proximally until the nosecone contacts a distal end of the second valve cover.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims. For example, although the retrieval catheter has been described herein for capturing a partially deployed prosthetic mitral valve, the retrieval catheter may also be used to capture another partially deployed prosthetic heart valve, such as a prosthetic aortic valve, tricuspid valve or pulmonary valve, or another implantable medical device.

1. A retrieval catheter for retrieving a partially deployed medical device held at the distal end of a delivery catheter, the retrieval catheter comprising:
an inner tubular shaft engageable with the delivery catheter in a manner that prevents translation of the delivery catheter in a longitudinal direction relative to the inner tubular shaft absent rotation of the delivery catheter relative to the inner tubular shaft, the inner tubular shaft having a proximal end, a distal end, and a lumen extending therethrough from the proximal end to the distal end, an outer surface in a distal portion of the inner tubular shaft having a series of outer threads;
an intermediate tubular shaft assembled around the inner tubular shaft, the intermediate tubular shaft having a proximal end, a distal end, and an inner surface defining a lumen therethrough from the proximal end to the distal end, the inner surface in a distal portion of the intermediate tubular shaft having a series of inner threads engageable with the outer threads of the inner tubular shaft such that rotation of the intermediate tubular shaft relative to the inner tubular shaft translates the intermediate tubular shaft proximally or distally relative to the inner tubular shaft and the delivery catheter;
an outer tubular shaft assembled around the intermediate tubular shaft, the outer tubular shaft having a proximal end, a distal end, and a lumen therethrough from the proximal end to the distal end; and
a valve cover at the distal end of the outer tubular shaft, the valve cover being operatively connected to the intermediate tubular shaft such that translation of the intermediate tubular shaft translates the valve cover.

2. The retrieval catheter as claimed in claim 1, wherein the distal portion of the inner tubular shaft is flexible.

3. The retrieval catheter as claimed in claim 2, wherein the distal portion of the inner tubular shaft includes a flexible distal section, a flexible proximal section, and a rigid tubular portion connected between the distal section and the proximal section.

4. The retrieval catheter as claimed in claim 3, wherein an inner surface of the rigid tubular portion includes a first plurality of threads.

5. The retrieval catheter as claimed in claim 4, wherein an outer surface of the delivery catheter includes a section having a second plurality of threads that are complementary to the first plurality of threads, whereby the engagement of the second plurality of threads with the first plurality of threads prevents the translation of the delivery catheter in the longitudinal direction relative to the inner tubular shaft absent rotation of the delivery catheter relative to the inner tubular shaft.

6. The retrieval catheter as claimed in claim 1, wherein the intermediate tubular shaft is flexible.

7. The retrieval catheter as claimed in claim 6, wherein the intermediate tubular shaft has a tri-coil structure.

8. The retrieval catheter as claimed in claim 6, wherein the intermediate tubular shaft is formed from a hypotube having a plurality of circumferential slits.

9. The retrieval catheter as claimed in claim 1, wherein the outer tubular shaft is flexible.

10. The retrieval catheter as claimed in claim 9, wherein the outer tubular shaft has a tri-coil structure.

11. The retrieval catheter as claimed in claim 9, wherein the outer tubular shaft is formed from a hypotube having a plurality of circumferential slits.

12. The retrieval catheter as claimed in claim 1, wherein the series of outer threads includes a plurality of turns of a helical coil fixedly connected to the outer surface in the distal portion of the inner tubular shaft.

13. The retrieval catheter as claimed in claim 12, wherein the series of inner threads includes a plurality of turns of a helical coil fixedly connected to the inner surface in the distal portion of the intermediate tubular shaft.

14. The retrieval catheter as claimed in claim 1, wherein the valve cover has a proximal end connected to the outer tubular shaft, an open distal end and an annular ring positioned inside the valve cover so as to define an annular pocket between the annular ring and the proximal end of the valve cover.

15. The retrieval catheter as claimed in claim 14, wherein the distal end of the intermediate tubular shaft includes an annular flange, the annular flange being captured within the annular pocket in the valve cover so that the intermediate tubular shaft is rotatable relative to the outer tubular shaft and not translatable relative to the outer tubular shaft.

16. The retrieval catheter as claimed in claim 1, wherein the valve cover has a proximal end connected to the outer tubular shaft and an open distal end, the valve cover at the open distal end flaring outwardly.

17. The retrieval catheter as claimed in claim 1, wherein the valve cover has a proximal end connected to the outer tubular shaft and an open distal end, the open distal end having a rounded edge.

18. A method for retrieving a partially deployed medical device held at a distal end of a catheter assembly within a patient, the catheter assembly including an outer sheath having a proximal end and a distal end, a first valve cover at the distal end of the outer sheath, a steering catheter disposed within the outer sheath, an extension catheter disposed within the steering catheter, and a suture catheter disposed within the extension catheter, the medical device being positioned outside of the valve cover and connected to the suture catheter by a plurality of tethers, the method comprising:
removing the extension catheter, the steering catheter, the outer sheath, and the valve cover from the catheter assembly and from the patient;
assembling a retrieval catheter over the suture catheter within the patient, the retrieval catheter including:
an inner tubular shaft engageable with the suture catheter in a manner that prevents translation of the suture catheter in a longitudinal direction relative to the inner tubular shaft;
an intermediate tubular shaft assembled around the inner tubular shaft, the intermediate tubular shaft being threadedly engaged with the inner tubular shaft;
an outer tubular shaft assembled around the intermediate tubular shaft; and
a second valve cover at the distal end of the outer tubular shaft, the second valve cover being operatively connected to the intermediate tubular shaft;
rotating the intermediate tubular shaft relative to the inner tubular shaft to translate the intermediate tubular shaft and the second valve cover distally relative to the suture catheter and the medical device until the second valve cover collapses and covers the medical device; and
removing the retrieval catheter, the suture catheter, and the medical device from the patient.

19. The method as claimed in claim 18, wherein the step of assembling the retrieval catheter over the suture catheter includes threadedly engaging the inner tubular shaft to the suture catheter.

20. The method as claimed in claim 18, wherein the catheter assembly further includes a nosecone catheter extending through the suture catheter and the medical device, and a nosecone on a distal end of the nosecone catheter, the nosecone being positioned distally of the medical device, the method further comprising:
after the second valve cover covers the medical device and before the removing step, retracting the nosecone catheter proximally until the nosecone contacts a distal end of the second valve cover.
